SEQUENCE LISTING (91 sequences)

SEQ ID NO: 1 - protein, 11 aa, Mus sp.
HRAERPFPEED

SEQ ID NO: 2 - protein, 9 aa, Mus sp.
LTPSFPVSP

SEQ ID NO: 3 - protein, 11 aa, Mus sp.
RVGGHLRLSGQ

SEQ ID NO: 4 - protein, 10 aa, Mus sp.
GVRAALPTPR

SEQ ID NO: 5 - protein, 11 aa, Mus sp.
QAGPVLPTSLE

SEQ ID NO: 6 - protein, 9 aa, Mus sp.
LMYRFEEEL

SEQ ID NO: 7 - protein, 11 aa, Mus sp.
WENVSPELNST

SEQ ID NO: 8 - protein, 9 aa, Mus sp.
SVYDFFVWL

SEQ ID NO: 9 - protein, 17 aa, Mus sp.
ISQAVHAAHAEINEAGR

SEQ ID NO: 10 - protein, 15 aa, Homo sapiens
TRFQTLLALHRSYLT

SEQ ID NO: 11 - protein, 19 aa, Homo sapiens
SFTRGVYYPDKVFRSSVLH

SEQ ID NO: 12 - protein, 24 aa, Homo sapiens
RKSNLKPFERDISTEIYQAGSTPC

SEQ ID NO: 13 - protein, 18 aa, Homo sapiens
TQLNRALTGIAVEQDKNT

SEQ ID NO: 14 - protein, 20 aa, Homo sapiens
GFNFSQILPDPSKPSKRSFI

SEQ ID NO: 15 - protein, 18 aa, Homo sapiens
IQDSLSSTASALGKLQDV

SEQ ID NO: 16 - protein, 24 aa, Homo sapiens
GKLQDVVNQNAQALNTLVKQLSSN

SEQ ID NO: 17 - protein, 21 aa, Homo sapiens
NAQALNTLVKQLSSNFGAISS

SEQ ID NO: 18 - protein, 18 aa, Homo sapiens
TAPAICHDGKAHFPREGV

SEQ ID NO: 19 - protein, 15 aa, Homo sapiens
GTHWFVTQRNFYEPQ

SEQ ID NO: 20 - protein, 16 aa, Homo sapiens
EPQIITTDNTFVSGNC

SEQ ID NO: 21 - protein, 22 aa, Homo sapiens
TTLDSKTQSLLIVNNATNVVIK

SEQ ID NO: 22 - protein, 14 aa, Homo sapiens
PFLGVYYHKNNKSW

SEQ ID NO: 23 - protein, 18 aa, Homo sapiens
PTESIVRFPNITNLCPFG

SEQ ID NO: 24 - protein, 16 aa, Homo sapiens
IPTNFTISVTTEILPV

SEQ ID NO: 25 - protein, 10 aa, Homo sapiens
YLAMPFATPM

SEQ ID NO: 26 - protein, 10 aa, Homo sapiens
LLMWITQCFL

SEQ ID NO: 27 - protein, 9 aa, Homo sapiens
SLLMWITQC

SEQ ID NO: 28 - protein, 11 aa, Homo sapiens
SLLMWITQCFL

SEQ ID NO: 29 - protein, 13 aa, Homo sapiens
APRGPHGGAASGL

SEQ ID NO: 30 - protein, 9 aa, Homo sapiens
LEFYLAMPF

SEQ ID NO: 31 - protein, 9 aa, Homo sapiens
FATPMEAEL

SEQ ID NO: 32 - protein, 9 aa, Homo sapiens
EADPTGHSY

SEQ ID NO: 33 - protein, 9 aa, Homo sapiens
NYKHCFPEI

SEQ ID NO: 34 - protein, 9 aa, Homo sapiens
KVLEYVIKV

SEQ ID NO: 35 - protein, 11 aa, Homo sapiens
QLVFGIELMEV

SEQ ID NO: 36 - protein, 9 aa, Homo sapiens
EVDPIGHLY

SEQ ID NO: 37 - protein, 11 aa, Homo sapiens
IMPKAGLLIIV

SEQ ID NO: 38 - protein, 9 aa, Homo sapiens
IMPKAGLLI

SEQ ID NO: 39 - protein, 9 aa, Homo sapiens
FLWGPRALV

SEQ ID NO: 40 - protein, 9 aa, Homo sapiens
KVAELVHFL

SEQ ID NO: 41 - protein, 9 aa, Homo sapiens
NLTHVLYPV

SEQ ID NO: 42 - protein, 9 aa, Homo sapiens
LYVDSLFFL

SEQ ID NO: 43 - protein, 9 aa, Homo sapiens
VLDGLDVLL

SEQ ID NO: 44 - protein, 10 aa, Homo sapiens
SLYSFPEPEA

SEQ ID NO: 45 - protein, 10 aa, Homo sapiens
ALYVDSLFFL

SEQ ID NO: 46 - protein, 9 aa, Homo sapiens
SLLQHLIGL

SEQ ID NO: 47 - protein, 15 aa, Homo sapiens
AAEIRASANLAATKM

SEQ ID NO: 48 - protein, 15 aa, Homo sapiens
AQKFNGLTVLPPLLT

SEQ ID NO: 49 - protein, 15 aa, Homo sapiens
ATRFASVYAWNRKRI

SEQ ID NO: 50 - protein, 15 aa, Homo sapiens
DEMIAQYTSALLAGT

SEQ ID NO: 51 - protein, 15 aa, Homo sapiens
DLFLPFFSNVTWFHA

SEQ ID NO: 52 - protein, 15 aa, Homo sapiens
EDLLFNKVTLADAGF

SEQ ID NO: 53 - protein, 15 aa, Homo sapiens
FFSNVTWFHAIHVSG

SEQ ID NO: 54 - protein, 15 aa, Homo sapiens
FGEVFNATRFASVYA

SEQ ID NO: 55 - protein, 15 aa, Homo sapiens
FSTFKCYGVSPTKLN

SEQ ID NO: 56 - protein, 15 aa, Homo sapiens
GSNVFQTRAGCLIGA

SEQ ID NO: 57 - protein, 15 aa, Homo sapiens
GWTFGAGAALQIPFA

SEQ ID NO: 58 - protein, 15 aa, Homo sapiens
IIAYTMSLGAENSVA

SEQ ID NO: 59 - protein, 15 aa, Homo sapiens
ITRFQTLLALHRSYL

SEQ ID NO: 60 - protein, 15 aa, Homo sapiens
KRSFIEDLLFNKVTL

SEQ ID NO: 61 - protein, 15 aa, Homo sapiens
LQIPFAMQMAYRFNG

SEQ ID NO: 62 - protein, 15 aa, Homo sapiens
NCTFEYVSQPFLMDL

SEQ ID NO: 63 - protein, 15 aa, Homo sapiens
NQKLIANQFNSAIGK

SEQ ID NO: 64 - protein, 15 aa, Homo sapiens
NSVAYSNNSIAIPTN

SEQ ID NO: 65 - protein, 15 aa, Homo sapiens
NYNYLYRLFRKSNLK

SEQ ID NO: 66 - protein, 15 aa, Homo sapiens
PIGINITRFQTLLAL

SEQ ID NO: 67 - protein, 15 aa, Homo sapiens
PINLVRDLPQGFSAL

SEQ ID NO: 68 - protein, 15 aa, Homo sapiens
PTNFTISVTTEILPV

SEQ ID NO: 69 - protein, 15 aa, Homo sapiens
QMAYRFNGIGVTQNV

SEQ ID NO: 70 - protein, 15 aa, Homo sapiens
QPYRVVVLSFELLHA

SEQ ID NO: 71 - protein, 15 aa, Homo sapiens
QQLIRAAEIRASANL

SEQ ID NO: 72 - protein, 15 aa, Homo sapiens
QSLLIVNNATNVVIK

SEQ ID NO: 73 - protein, 15 aa, Homo sapiens
QTYVTQQLIRAAEIR

SEQ ID NO: 74 - protein, 15 aa, Homo sapiens
REGVFVSNGTHWFVT

SEQ ID NO: 75 - protein, 15 aa, Homo sapiens
RGVYYPDKVFRSSVL

SEQ ID NO: 76 - protein, 15 aa, Homo sapiens
SEFRVYSSANNCTFE

SEQ ID NO: 77 - protein, 15 aa, Homo sapiens
SFVIRGDEVRQIAPG

SEQ ID NO: 78 - protein, 15 aa, Homo sapiens
SSGWTAGAAAYYVGY

SEQ ID NO: 79 - protein, 15 aa, Homo sapiens
TGRLQSLQTYVTQQL

SEQ ID NO: 80 - protein, 15 aa, Homo sapiens
TLLALHRSYLTPGDS

SEQ ID NO: 81 - protein, 15 aa, Homo sapiens
TSNFRVQPTESIVRF

SEQ ID NO: 82 - protein, 15 aa, Homo sapiens
TVEKGIYQTSNFRVQ

SEQ ID NO: 83 - protein, 15 aa, Homo sapiens
TWRVYSTGSNVFQTR

SEQ ID NO: 84 - protein, 15 aa, Homo sapiens
VKQLSSNFGAISSVL

SEQ ID NO: 85 - protein, 15 aa, Homo sapiens
VLSFELLHAPATVCG

SEQ ID NO: 86 - protein, 15 aa, Homo sapiens
VNFNFNGLTGTGVLT

SEQ ID NO: 87 - protein, 15 aa, Homo sapiens
YFKIYSKHTPINLVR

SEQ ID NO: 88 - protein, 15 aa, Homo sapiens
YRLFRKSNLKPFERD

SEQ ID NO: 89 - protein, 15 aa, Homo sapiens
YSVLYNSASFSTFKC

SEQ ID NO: 90 - protein, 15 aa, Homo sapiens
YTSALLAGTITSGWT

SEQ ID NO: 91 - protein, 15 aa, Homo sapiens
YYVGYLQPRTFLLKY

(19) United States
(12) Patent Application Publication
(10) Pub. No.: US 2023/0132140 A1
(43) Pub. Date: Apr. 27, 2023

(54) METHOD OF TREATING CANCER

(71) Applicant: Celloram Inc., Cleveland, OH (US)

(72) Inventors: Seunghwan Lim, Solon, OH (US); Tej Kumar Pareek, Beachwood, OH (US); Liraz Levi, Pepper Pike, OH (US); Seong-Jin Kim, Seoul (KR)

(73) Assignee: Celloram Inc., Cleveland, OH (US)

(21) Appl. No.: 17/945,468

(22) Filed: Sep. 15, 2022

Related U.S. Application Data
(60) Provisional application No. 63/244,533, filed on Sep. 15, 2021.

(51) Int. Cl. A61K 39/00; A61P 35/00

(52) U.S. Cl. CPC A61K 39/001129; A61P 35/00; A61K 39/001184; A61K 2039/5158; A61K 2039/55516; A61K 2039/5154

(57) ABSTRACT

The present application discloses a method of treating cancer, including administering to a person suffering from cancer or in remission from cancer, an antigen presenting cell loaded with an immunogenic CD4 T cell activating antigen and a CD8 T cell activating neoantigen specific for the cancer.

### BACKGROUND OF THE INVENTION

### 1. Field of the Invention

The present application relates to a method of treating or preventing cancer. The application also relates to use of vaccine to cancer to treat or prevent severity of cancer progression.

### 2. General Background and State of the Art

The arrival of immunotherapy has brought fresh attention to the role that vaccines can play in stimulating the body's natural defenses against the abnormal cell growth that leads to malignancies. Vaccines are currently in limited use to prevent viral based cancers like HPV, but the real promise lies in their potential in treating and fighting recurrence for patients already diagnosed with the disease.

In cancer immunotherapy, CD4+ T cells are known to play a key role in recruitment and activation of CD8+T cells by direct interaction and/or by ‘licensing’ dendritic cells through which the efficacy and strength of antitumor immunity are enhanced. Thus, exploiting CD4+T cells for induction of more efficient antitumor immune responses has been a major focus of research, but it has been very challenging due to lack of availability of identifying CD4+T specific cancer epitopes with currently existing screening algorithms.

To overcome these limitations, the product of the present invention (hereafter PROTEXI) functionally harnesses CD4+T ‘helper’ to recruit and activate CD8+T tumor-killing cells by co-presenting non-tumor-specific but highly immunogenic CD4+T epitope such as Spike epitopes of Coronavirus, and other epitopes of viral or bacterial origin, and tumor-specific CD8+T neoepitopes or tumor-associated antigens (e.g. cancer/testis antigens (CTAs)) simultaneously on antigen presenting cells such as dendritic cells.

### SUMMARY OF THE INVENTION

The present invention is directed to a method of treating cancer, comprising administering to a person suffering from cancer or in remission, an antigen presenting cell loaded with an immunogenic CD4+ T cell activating antigen and a CD8+ T cell activating neoantigen specific for the cancer. The antigen presenting cell may be a dendritic cell. The cell may be autologous. The CD4 T cell activating antigen may be a peptide. The peptide may be a fragment of a pathogen, or epitope fragments used for preventive vaccination throughout lifetime. The pathogen may be a bacteria, virus, or parasite. The virus may be a coronavirus, Influenza, Mycobacterium tuberculosis, Cytomegalovirus (CMV). The peptide may be a fragment of spike protein, ORF3a, ORF7a, ORF6, ORF8, nsp2, nsp5 of coronavirus, HA of influenza, GlfT2, fas, fbpA, iniB, PPE15 of M. tuberculosis, pp50, pp65, IE-1, gB, gH of CMV. The CD8 T cell activating neoantigen may be a publicly known neoantigen. The neoantigen may be any peptide from Tables 4 to 7. In particular, the neoantigen may be personalized neoantigen or public/shared tumor-specific antigen including cancer/testis antigens, repetitive elements, and transposable elements. The cancer may be prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In particular, the cancer may be sarcoma and further in particular, the cancer may be osteosarcoma.

In another aspect, the invention is directed to a method of enhancing anti-tumor immunity of a person in remission of cancer, comprising administering to the person an antigen presenting cell loaded with an immunogenic CD4+ T cell activating antigen and a CD8+ T cell activating neoantigen specific for the cancer. The antigen presenting cell may be a dendritic cell. The cell may be autologous. The CD4+ T cell activating antigen may be a peptide. The peptide may be a fragment of a pathogen, or epitope fragments used for preventive vaccination throughout lifetime. The pathogen may be a bacteria, virus, or parasite. The virus may be a coronavirus, Influenza, Mycobacterium tuberculosis, Cytomegalovirus (CMV). The peptide may be a fragment of spike protein, ORF3a, ORF7a, ORF6, ORF8, nsp2, nsp5 of coronavirus, HA of influenza, GlfT2, fas, fbpA, iniB, PPE15 of M. tuberculosis, pp50, pp65, IE-1, gB, gH of CMV. The CD8+ T cell activating neoantigen may be a publicly known neoantigen. The neoantigen may be any peptide from Tables 4 to 7. In particular, the neoantigen may be personalized neoantigen or public/shared tumor-specific antigen including cancer/testis antigens, repetitive elements, and transposable elements. The cancer may be prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof. In particular, the cancer may be sarcoma and further in particular, the cancer may be osteosarcoma.

In yet another aspect, the invention is directed to a cancer vaccine, comprising an antigen-presenting cell co-presenting a non-tumor-specific, but highly immunogenic CD4+ T cell epitope and tumor-specific CD8+ T cell neoepitopes simultaneously, which empowers antitumor immune response by engaging CD4+ T cells for activation of CD8+ T cells. The vaccine is autologous with respect to the subject treated. The antigen presenting cell is a dendritic cell. The CD4+ T cell epitope derives from bacteria or virus. The CD4+ T cell epitope is spike protein from coronavirus.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1F show that PROTEXI enhanced immune responses rejecting F420 osteosarcoma in syngeneic mouse model. (A) Experimental plan with three treatment groups, control, PROTEXI, and PROTEXI with 2-times of pre-vaccination (DC_{OVA323}) at −14 d and −7 d. The PROTEXI vaccination conducted at d0, d7, and d14(1×10⁶/inj. SQ). The PROTEXI is prepared by pulsing matured bone-marrow-derived dendritic cells (BMDC) with OVA323-339 peptide (5 μg/ml) and neoantigens (MT4-1, 4-2, 4-6, 4-7, 4-8, 2 μg/ml each as shown in table). (B) The bioluminescence images of treatment groups at day 16 are shown on top and the increase of tumor burden is marked as total flux (bottom). (C) The tumor tissues were harvested at day 21 and subjected to immunostaining (anti-CD3 antibody) for the comparison of CD3+T tumor infiltrating leukocytes and the representative images are shown. (D) The number of CD3+T lymphocytes were counted in the 5 random field per tumor section using Image J software. (E) The draining lymph nodes and spleen are harvested to measure the level of cytotoxic T cells (CD8+, IFN-γ+) and regulatory T cell (Treg, CD4+, CD25+, FoxP3+) in the cohorts. (F) ELISPOT (IFN-γ) assay performed for antigen-specific T cell detection. Splenocytes of treated group are co-cultured with mature BMDC pulsed with NeoAg (MT4-1, 4-2, 4-6, 4-7, 4-8) or OVA323 as indicated. Splenocyte of each without antigenic peptide were used as control. The number of IFN-γ+ T cells are counted using ELISPOT counter from Cellular Technology Limited. ** p<0.01, ****<0.0001

FIGS. 2A-2C show that PROTEXI vaccination rejected B16F10 melanoma in syngeneic mouse model. (A) Experimental plan with three treatment groups, control, PROTEXI, and PROTEXI with pre-vaccination (DC_{OVA323}) at −14 d. The PROTEXI vaccination conducted at d0, and d14(1×10⁶/inj. SQ) following the B16F10 injection S.Q. at d0. (B) The PROTEXI is prepared by pulsing matured BMDC with OVA323 (5 μg/ml) and tumor-associated antigens (TAAs) (M30-11 and Trp2, 2 μg/ml each as shown in table) while DC_{TAA} was pulsed only with TAAs only (M30-11, Trp2). (C) After injection, the B16F10 tumor growth was monitored by measuring tumor size in every 2-3 days. Measured tumor length (L) and width (W) using caliper and then calculate tumor volume using formulations V=(L×W×W)/2. * p<0.05

FIGS. 3A-3J show that combination of OTII-CD4T and PROTEXI enhanced immune rejection of B16F10 melanoma (A) Experimental plan with treatment groups, consisted of DC_{TAA}, DC_{OVA323}, and PROTEXI with OTII-CD4T co-administration and control with B16F10 injection only. The vaccination (1×10⁶/inj. SQ) conducted only one time at d0 and followed by monitoring tumor growth. (B) The table shows the peptide-pulsed DC vaccine and OVA₃₂₃ epitope-specificity of OTII-CD4T cell. (C) The tumor volume of individual mouse was shown in each group (n=4-5). (D) The average of tumor growth in each group is compared over time up to day 36. * p<0.05. (E, F) Emergence of antigen-specific T cells clones in the spleen was tested by conducting ELISPOT (IFN-γ) assay. The indicated peptides (2 μg/ml) added to the splenocytes (5×10⁶/well) isolated from the vaccinated mice for in vitro stimulation (IVS) and maintained in culture for 2 weeks. Then, the splenocytes (3×10⁴/well) were subjected to ELISPOT assay and counted the IFN-γ spots using ELISPOT reader (CTL). **p<0.01. (G) The level of epitope-specific TCR+ cells in the IVS splenocytes were measured by flow cytometry gating live, singlet, Trp2 or Luc2 peptide-loaded tetramer+ and anti-CD8+ cells. (H) The percent of CD8+ TCR+ T cells specifically binding to tetramer loaded with Trp2 and Luc2 epitope were compared among the treated groups. (I) The tumor staining was performed to compare the TIL (CD4+, CD8+, CD11c+(DC), Pink color) among the groups and the overall structure of tumors were visualized with H&E staining. The scale bar is shown at the corner. (J) The tumor infiltrated immune cells (CD4T, CD8 T, and CD11c) were counted in 10 random field of three tumors (30 areas total) in each group using Image J software. One-way ANOVA Tukey's multiple comparisons test, ****<0.0001

FIG. 4A-4D show that CD4 T cell depletion abrogated PROTEXI-mediated tumor rejection in B16F10-T1 model (A) B16F10-T1 (1×10⁵/inj.), a highly aggressive B16F10 established via in vivo selection, injected into three treatment groups (n=5/group) (1) control+PBS, (2) IgG+ PreVax(DC_{OVA323})+PROTEXI, and (3) αCD4+ PreVax(DC_{OVA323})+PROTEXI. The CD4 T cell depletion was induced by injecting αCD4 antibody (250 μg/inj.) or IgG at −d10 and −d7 followed by pre-vaccination with DC_{OVA323} (1×10⁶/inj.) at −d7. The PROTEXI (1×10⁶/inj.) was administered at d0. (B) The tumor sizes were measured at 3-day interval up to d19 post-tumor injection. (C) Splenocytes (n=3/group) from each cohort were subjected to in vitro stimulation (IVS) with peptide epitopes, OVA323, Trp2 in the culture media supplemented with IL-7 (20 ng/ml) for initial 3 days followed by IL-2 (20 ng/ml). After 7 days of IVS, the population of CD4 and CD8 T cells was determined by flow cytometry. (D) CD4 T cell depletion was effective even after 7 day IVS culture.

FIG. 5A-5B show that PROTEXI-induced Trp2-specific CD8T cell activation is principally depending on CD4 helper T cells. (A) The SP-T cells of each group with 1 week-IVS were re-challenged with mDC loaded OVA₃₂₃/Trp2 peptide (2 μg/ml) for 1 hour followed by Golgi-stop for 4 hours. The Trp2-specific CD8 T cell activity was determined by the level of IFNγ, TNFα, IL-2 production in each group. The representative dot plots of CD8+ T cells are shown. (B) The OVA₃₂₃-specific CD4 T and Trp2-specific CD8 T cells were highly activated only in the IgG-treated group.

FIG. 6A-6B show that CD4 T depletion resulted in loss of T cell memory cell formation induced by PROTEXI. (A) The CD4 and CD8 T memory cells were compared by demonstrating CD44 and CD62L positive populations by flow cytometry. Naïve memory T cell (T_{M} naïve, CD44⁻CD62L⁺), central memory T cell (T_{CM}, CD44⁻CD62L⁺), effector memory T cell (T_{EM}, CD44⁺CD62L⁻). (B) CD4 and CD8 effector memory T cells were exclusively increased in the IgG+PROTEXI group.

FIGS. 7A-7F show that PROTEXI robustly inhibited F420 growth by exploiting Spike epitope-specific human CD4T cells. (A) The humanized mice, referred as DR4 mice, possesses genomic background with mouse MHC-II (I-A) knockout, a chain fusion protein of HLA-DRA extracellular domain and mouse I-E intracellular domain, β chain fusion protein of HLA-DRB*0401 extracellular domain and mouse I-E as shown in the drawing. (B) The treatment plan with two times of PROTEXI vaccination with or without PreVax (S236) at −d7. PROTEXI vaccination carried out at day 0 and day 7 following F420(1×10⁶/inj) injection at day 0. (C) The immunogenic epitopes used in the vaccination. MT4-1, 4-2, 4-4, 4-6, 4-7, 4-8 NeoAgs are screened by Deepomics algorithm but displayed poorly immunogenic characteristics. The MHC-II restricted Spike S236-250 peptide was reported to be able to induce T cell responses in the COVID-19 patient's PBMC. (D) After the vaccination, F420 tumor growth was monitored by measuring tumor size and calculated the tumor volume. Control (blue), PROTEXI only (red), PreVax+PROTEXI (green) (E, F) it is compared the ratio of CD4, CD8 T cells, CD8T (IFN-γ+) and Treg (CD25+FoxP3+) cells among treatment group in spleen and draining lymph nodes.

### DETAILED DESCRIPTION OF THE PREFERRED EMBODIMENTS

As used herein, and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like regarding the recitation of claim elements, or use of a “negative” limitation.

Certain features of the invention, which are described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

One aspect is a method of treating cancer in an individual, comprising recruiting a preexisting immune response in the body to a cancer site, thereby treating the cancer.

As used herein, cancer refers to diseases in which abnormal cells divide without the appropriate control of cell division and/or cellular senescence. The term cancer is meant to encompass solid tumors as well as blood borne cancer. Generally, a tumor is an abnormal mass of tissue that usually does not contain a cyst or liquid area. Solid tumors may be benign (not life threatening), or malignant (life threatening). Different types of solid tumors are named for the type of cells that form them. Examples of solid tumors include sarcomas, carcinomas, and lymphomas. Blood cancers (also called hematologic cancers) are cancers that begin in blood-forming tissue, such as the bone marrow, or in the cells of the immune system. Examples of blood cancer include leukemia, lymphoma, and multiple myeloma.

In some cancers, the cells can invade tissues other than those from which the original cancer cells arose. In some cancers, cancer cells may spread to other parts of the body through the blood and lymph systems. Thus, cancers are usually named for the organ or type of cell in which they start. For example, a cancer that originates in the colon is called colon cancer; cancer that originates in melanocytes of the skin is called melanoma, etc. As used herein, cancer may refer to carcinomas, sarcomas, adenocarcinomas, lymphomas, leukemias, etc., including solid and lymphoid cancers, gastric, kidney cancer, breast cancer, lung cancer (including non-small cell and small cell lung cancer), bladder cancer, colon cancer, ovarian cancer, prostate cancer, pancreatic cancer, stomach cancer, brain cancer, head and neck cancers, skin cancer, uterine cancer, testicular cancer, esophageal cancer, liver cancer (including hepatocarcinoma), lymphoma, including non-Hodgkin's lymphomas (e.g., Burkitt's, Small Cell, and Large Cell lymphomas) and Hodgkin's lymphoma, leukemia, and multiple myeloma. In exemplary embodiments, the cancer is lung cancer or adenocarcinoma.

As used herein, an immune, or immunological, response refers to the presence in an individual of a humoral and/or a cellular response to one or more antigens. For purposes of this disclosure, a “humoral response” refers to an immune response mediated by B-cells and antibody molecules, including secretory (IgA) or IgG molecules, while a “cellular response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity, of effector cells against cells displaying peptide antigens in association with MHC molecules on their surface. A cellular immune response also refers to the production of cytokines, chemokines and other such molecules produced by activated T-cells and/or other white blood cells, including those derived from CD4+ and CD8+T-cells.

Thus, an immunological response may be one that stimulates CTLs, and/or the production or activation of helper T-cells. The production of chemokines and/or cytokines may also be stimulated. The immune response may also comprise an antibody-mediated immune response. Hence, an immunological response may include one or more of the following effects: the production of antibodies (e.g., IgA or IgG) by B-cells; and/or the activation of suppressor, cytotoxic, or helper T-cells, and/or T-cells directed specifically to an antigen. Such responses can be determined using standard immunoassays and neutralization assay, known in the art.

As used herein, a “preexisting immune response” is an immune response that is present in an individual prior to initiation of the cancer treatment. Thus, an individual having a preexisting immune response has an immune response against an antigen, prior to the initiation of a treatment using the antigen to treat cancer. A preexisting immune response can be a naturally occurring immune response, or it can be an induced immune response. As used herein, a naturally occurring preexisting immune response is an immune response in an individual that was elicited in response to an antigen, such as a bacterial or viral antigen, which the individual came into contact with intentionally or unintentionally. An induced preexisting immune response is an immune response resulting from intentional exposure to an antigen, such as when receiving a vaccine. The preexisting immune response may be a naturally-occurring immune response, or the preexisting immune response may be an induced immune response.

As used herein, the phrase “recruiting an immune response,” refers to a process in which an antigen is administered in the form of PROTEXI to an individual such that components of a preexisting immune response travel through the body to the location where the antigen/PROTEXI was administered, resulting in attack by the immune system components on cancer cells displaying neoantigen.

As used herein, the phrase “treating a cancer” refers to various outcomes regarding a cancer. Treating a cancer includes reducing the rate of increase in the number of cancer cells in a treated individual. Such a reduction in the rate of increase can be due to a slowing in replication of cancer cells. Alternatively, the replication rate of cancer cells may be unaffected, an increase in the number of cancer cells may be killed by the preexisting immune response. In certain aspects, treating a cancer refers to a situation in which the number of cancer cells stops increasing, but remains at a constant level. Such a situation may arise due to inhibition of cancer cell replication by recruitment of the preexisting immune response, or it may be due to the rate of production of new cancer cells being balanced by the rate of cancer cell killing by the recruited preexisting immune response. Treating a cancer refers to stabilizing the cancer such that the growth of the cancer is decreased or stopped, or a decrease in the number of cancer cells in the treated individual, and/or in the individual being cancer free (i.e., no detectable cancer cells).

As used herein, “cancer vaccines” may include various compositions that contain tumor associated antigens (or which can be used to generate the tumor associated antigen in the subject) and thus can be used to provoke an immune response in a subject that will be directed to tumor cells that contain the tumor associated antigen. Conventionally known example of cancer vaccine include, attenuated cancerous cells, tumor antigens, antigen presenting cells such as dendritic cells pulsed with tumor derived antigen or nucleic acids encoding tumor associated antigens. In some embodiments, a cancer vaccine may be prepared with a patient's own cancer cells. In some embodiments, a cancer vaccine may be prepare with biological material that is not from a patient's own cancer cells.

As used herein, “DC-” with a hyphen before or after refers to dendritic cell loaded with the antigen indicated before or after the hyphen. For example, DC-TAA or TAA-DC means dendritic cells loaded with TAA. Likewise, “DC” with an antigen in subscript after “DC” refers to dendritic cell loaded with the antigen indicated after the DC and in the subscript. For example, DC_{TAA} means dendritic cells loaded with TAA.

As used herein, “DC-MI”, refers to dendritic cell that is loaded with an antigen restricted by MHC-I, such as tumor associated antigen (TAA).

As used herein, “DC-MII”, refers to dendritic cell that is loaded with an antigen restricted by MHC-II, such as pathogen associate antigen (PAA).

As used herein, “PROTEXI” refers to a DC (dendritic cell) vaccine platform co-presenting highly immunogenic MHC-II restricted antigen and MHC-I neoAgs that boosts cytotoxic T cell response to malignant tumors. Without limitation, the DC may be autologous. In essence, PROTEXI is “MI-DC-MII”.

PROTEXI are dendritic cells used in an immunotherapeutic mechanism which leverages an epitope-specific CD4⁺ T cells such as Spike protein from Coronavirus for purposed epitope spreading to NeoAg-specific CD8⁺ T cells to ultimately offer highly effective, long-lasting immune responses to cancer patients such as AYA osteosarcoma patients, in particular, those cancer patients who are previously infected or vaccinated for SARS-CoV-2. In this regard, PROTEXI comprises dendritic cells on which are loaded neoantigens associated with MHC-I and a peptide associated with MHC-II. The neoantigens may be publicly known or specific to an individual such as seen in autologous dendritic cells. The peptide associated with MHC-II may be a CD4+ T cell activating peptide, and may be without limitation, a pathogenic peptide.

As used herein, “MHC-I” means major histocompatibility complex class I molecule.

As used herein, “MHC-II” means major histocompatibility complex class II molecule.

As used herein, “mDC” means mature dendritic cell.

As used herein, “neoantigen” means tumor-specific antigens generated by mutations in tumor cells, which are expressed only in tumor cells and never recognized by immune cells before.

As used herein, “epitope spreading” means enhancement and diversification of T cell response to targeted epitopes as well as the other epitopes originated endogenously from tumors or pathogens.

As used herein, “personalized pharmaceutical” refers to specifically tailored therapies for one individual patient that will only be used for therapy in such individual patient, including actively personalized cancer vaccines and adoptive cellular therapies using autologous patient tissue.

As used herein, “pharmaceutical composition” refers to a composition suitable for administration to a human being in a medical setting. Preferably, a pharmaceutical composition is sterile and produced according to GMP guidelines.

As used herein, “pre-vaccination” means induction of antigen-specific T cell immunity prior to tumor challenge and sometimes referred to as Pre-Vax where the Vax refers to vaccination. The induction may be intentional or unintentional. In other words, pre-vaccination may occur intentionally by injecting a person with T cell immunity inducing material for the purpose and goal of treating cancer. Or, the induction may have occurred unintentionally caused by either infection with a virus or other microorganism, or by a vaccine administered not purposed for cancer treatment, but in which T cell immunity is induced.

As used herein “spike protein” or “spike glycoprotein” is the spike that studs the surface of the coronavirus, giving it the appearance of a crown to electron microscopy, hence “corona”.

As used herein, “sarcoma” includes bone sarcomas (osteosarcoma, chondrosarcoma, and Ewing's sarcoma) and soft-tissue sarcomas (leiomyosarcoma, synovial cell sarcoma, liposarcoma and so on).

Sarcomas significantly impact the adolescent and young adult (AYA) population and the prognosis for AYA patients with advanced disease is uniformly poor. Conventional therapies are effective for the early-stage disease but have limited utility in late-stage, metastatic sarcomas. An innovative therapeutic approach that overcomes the limitations of MHC-II neoantigen availability while leveraging CD4+ T function in a manner that promotes vigorous CD8+ T activation would be a leap forward in cancer vaccine development with the benefit of enhanced anti-tumor immune responses.

Sarcomas are a group of rare connective tissue cancers consisting of soft-tissue sarcomas (>50 subtypes, STS) and bone sarcomas (osteosarcoma[OS], chondrosarcoma[CS], and Ewing's sarcoma[ES]) [1, 2] with most occurring in fewer than 5 per 1,000,000. There is a notable increased rate of incidence for bone sarcomas in adolescent and young adult (AYA) patients, with a range of 400-1,000 new osteosarcoma cases each year in the United States. Conventional therapies (surgery, chemotherapy, radiotherapy) are effective for early-stage disease but once metastasized, the 5-year survival rate for metastatic osteosarcoma patients is only 30%. Thus, there is an urgent need for effective, novel therapies for sarcoma.

By “at risk of” is intended to mean at increased risk of, compared to a normal subject, or compared to a control group, e.g. a patient population. Thus, a subject carrying a particular marker may have an increased risk for a specific disease or disorder, and be identified as needing further testing. “Increased risk” or “elevated risk” mean any statistically significant increase in the probability, e.g., that the subject has the disorder. The risk is preferably increased by at least 10%, more preferably at least 20%, and even more preferably at least 50% over the control group with which the comparison is being made.

As used herein, “cell therapy” is also considered as ex vivo therapy, in that cells are grown or treated outside of the body and are then returned to the patient by injection or transplantation. The treated cells may be autologous or allogeneic relative to the patient.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

The term “transfection” is used herein to refer to the uptake of foreign DNA by a cell. A cell has been “transfected” when exogenous DNA has been introduced inside the cell membrane. A number of transfection techniques are generally known in the art. See, e.g., Graham et al. Virology, 52:456 (1973); Sambrook et al. Molecular Cloning, a laboratory manual, Cold Spring Harbor Laboratories, New York (1989); Davis et al., Basic Methods in Molecular Biology, Elsevier (1986), and Chu et al. Gene 13:197 (1981). Such techniques can be used to introduce one or more exogenous DNA moieties, such as a plasmid vector and other nucleic acid molecules, into suitable host cells. The term refers to both stable and transient uptake of the genetic material.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

As used herein, the term “administering,” refers to the placement of an agent or a composition as disclosed herein into a subject by a method or route which results in at least partial localization of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intravascular, intravenous, intraarterial, subarachnoid, subcapsular, subcutaneous, transmucosal, or transtracheal. Via the parenteral route, the agent or composition may be in the form of solutions or suspensions for infusion or for injection, or as lyophilized powders.

PROTEXI

The present invention relates to a novel method of treating cancer. Specifically, the present invention relates to a method of treating cancer in an individual, utilizing the individual's own immune system to attack cancer cells. The method makes use of the fact that individuals possess preexisting immune responses that were not originally elicited in response to a cancer, but that were elicited instead by microorganisms in the environment or a vaccine, such as the coronavirus and vaccine against it.

Multiple immunotherapeutic approaches including immune checkpoint blockade (ICB), adoptive T cell transfer (ACT), chimeric antigen receptor T cells (CAR-T), and cancer vaccines have significantly impacted survival for patients with many forms of cancer, and each of these forms of immunotherapy are currently under clinical evaluation for sarcomas. However, the successful application of immunotherapy in sarcomas has been hindered by the extensive heterogeneity of the disease, a limited understanding of immune response of sarcoma, and a lack of well characterized tumor-specific antigens for use in designing personalized ACT, CAR-T, and cancer vaccines[3]. The innovation and approach described in this application leverage three major observations that independently provide a foundation and important insights guiding the development of the proposed, unprecedented immunotherapeutic modality, designed to yield a highly effective and safe approach to achieve durable, relapse-free survival in sarcoma patients.

Firstly, CD4+ T helper cells play a central role during the induction of adaptive immune responses in the tumor microenvironment (TME) by “helping” tumor specific CD8+ cytotoxic T cell activation (CTL)[4]. CD4+ T cells can be activated by recognizing MHC-II loaded foreign antigens or ‘neo’ antigens (e.g. tumor-specific antigens never expressed in normal cells), which in turn recruit and orchestrate local immunity to fight against either infected or abnormal cancer cells. Although a range of CD8+ T cells can be activated by antigen presenting cells (APCs) or dendritic cells (DCs) loaded with MHC-I neoantigens, CD8+ T cell cytotoxicity against tumor is often transient and inefficient in memory T cell formation in the absence of CD4+ T helper cells. As such, the important role of CD4+ T cells has been demonstrated as essential to achieve strong anti-tumor immunity. However, the use of MHC-II restricted neoantigens has not been actively incorporated into tumor vaccination strategies in clinical trials due to the limitations of existing MHC-II prediction algorithms.

Secondly, one of the first pre-clinical studies has shown that the combined administration of T helper peptide and CTL epitopes exerted highly efficient protection in animals challenged with tumor [5]. Congruently, a recent study clearly showed that an optimal anti-tumor immune response requires the activity of both tumor-antigen-specific CD8+ T and CD4+ T cells where MHC-II restricted neoantigens expressed “simultaneously” with nonoverlapping MHC-I neoantigens by sarcomas (MHC-II null) play a pivotal role in promoting anti-tumor immunity [6]. These seminal, novel observations highlight the importance of presenting both MHC-I and MHC-II neoantigens “simultaneously”, through a process that is facilitated predominantly by DCs in the TME of sarcoma and that is ultimately essential for successful tumor immunity.

Thirdly, vaccine-induced epitope spreading is one of the key mechanisms for long-term disease-free survival in immune reactive cancer patients [7]. The delivery of CD4+ T cell helper signals is mediated by dendritic cells which play an important role for diversifying NeoAg-specific CTL responses via cross-priming and epitope spreading[7-9]. Through this mechanism, active CD4+ T cells further invigorate CD8+ T cell differentiation, optimize CTL memory formation, and ultimately contribute to the quality of CD8+ T cell responses. Although personalized tumor vaccination has been in the limelight recent years, an approach designed to specifically and purposely boost epitope spreading for the induction of highly efficient anti-tumor immunity is to our knowledge, unprecedented.

Since the World Health Organization (WHO) announced SARS-CoV-2 (COVID-19) as a pandemic in March 2020, COVID-19 vaccines have been rapidly developed and distributed worldwide in 2021 in a historically rapid timeframe. Strikingly, the most popular vaccines are developed in the novel platforms including mRNA-LNP and adenovirus-packaged vaccines that deliver the Spike protein sequences to prevent viral entry into human cells. The Spike protein-derived epitope(s) are pathogen associated antigens (PAA) that are highly immunogenic and induce robust CD4+ T cell activation, leading to CD8+ T reactivity and anti-Spike antibody production [10]. At the time of the present application, over 68% of the United States and 63% of the worldwide population are fully vaccinated for SARS-CoV-2. Preclinical studies show efficacy of our PROTEXI technology, a novel personalized, autologous dendritic cell (DC) vaccine which leverages the existence of Spike epitope-specific CD4+ T cells to promote the proposed “epitope spreading” to NeoAg-specific CD8+ T cells. The results validate a novel vaccine modality to potently induce tumor-specific immune responses in osteosarcoma patients previously infected or vaccinated for SARS-CoV-2. Since combination of nivolumab and ipilimumab showed a partial success for induction of better immune responses in the clinical trial (NCT02500797) compared to nivolumab monotherapy [11], a combination of PROTEXI-PAA and immune modulators (i.e. nivolumab and Vactosertib) would facilitate a significantly more effective and durable immunotherapeutic responses for AYA sarcoma patients. Vactosertib (IUPAC name for Vactosertib is 2-fluoro-N-[[5-(6-methylpyridin-2-yl)-4-([1,2,4]triazolo[1,5-a]pyridin-6-yl)-1H-imidazol-2-yl]methyl]aniline; CAS No.: 1352608-82-2).

In the present application is provided a description of the PROTEXI system for vaccination against cancer.

PROTEXI is an autologous, neoantigen (neoAgs) pulsed dendritic cell (DC) vaccine platform co-presenting MHC-II pathogen-associated antigens (PAA) along with MHC-I tumor neoAgs, which significantly enhances CD4+ T cell-mediated expansion of CD8+ T cells and thereby induces a more effective and durable immune response to cancer and in particular, sarcoma.

Vaccine

In general, vaccination is widely used as a method for preventing infectious diseases. Vaccination induces immune responses including T cell, B cell responses which are activated to target pathogens or abnormal cells. For immunotherapy of cancer, the term vaccination has been frequently used in the sense of induction of T cell immunity against cancer, in which abnormal cells express tumor-specific antigens. In this regard, the present invention is directed to prophylactic (preventive) and therapeutic vaccination methods using immunotherapy. In particular, when PROTEXI is administered to a person in whom immune response is already present, such as by being vaccinated with Spike peptide, such process may be thought of as therapeutic vaccination. For example, APCEDEN® is a personalized dendritic cell (DC)-based immunotherapy product that enhances antitumor immunity by ex vivo maturation of monocyte-derived DCs pulsed with whole tumor lysate [12, 13].

Antigens and Variants Thereof

The disclosed methods can also be practiced using one or more antigens, each of which independently comprises an amino acid sequence that is a variant of an at least 8 contiguous amino acid sequence. As used herein, a variant refers to a protein, or nucleic acid molecule, the sequence of which is similar, but not identical to, a reference sequence, wherein the activity (e.g., immunogenicity) of the variant protein (or the protein encoded by the variant nucleic acid molecule) is not significantly altered. These variations in sequence can be naturally occurring variations or they can be engineered using genetic engineering techniques known to those skilled in the art. Examples of such techniques are found in Sambrook J, Fritsch E F, Maniatis T et al., in Molecular Cloning-A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6.

Regarding variants, any type of alteration in the amino acid sequence is permissible so long as the resulting variant protein retains the ability to elicit an immune response. Examples of such variations include, but are not limited to, deletions, insertions, substitutions and combinations thereof. For example, with proteins it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, variant proteins can contain amino acid substitutions relative to a reference protein (e.g., wild-type protein). Any amino acid substitution is permissible so long as the activity of the protein is not significantly affected. In this regard, it is appreciated in the art that amino acids can be classified based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Naturally occurring residues may be divided into classes based on common side chain properties: 1) hydrophobic: Met, Ala, Val, Leu, Ile; 2) neutral hydrophilic: Cys, Ser, Thr; 3) acidic: Asp, Glu; 4) basic: Asn, Gln, His, Lys, Arg; 5) residues that influence chain orientation: Gly, Pro; and 6) aromatic: Trp, Tyr, Phe.

For example, non-conservative substitutions may involve the exchange of a member of one of these classes for a member from another class.

In making amino acid changes, the hydropathic index of amino acids may be considered. Each amino acid has been assigned a hydropathic index based on its hydrophobicity and charge characteristics. The hydropathic indices are: isoleucine (+4.5); valine (+4.2); leucine (+3.8); phenylalanine (+2.8); cysteine/cystine (+2.5); methionine

(+1.9); alanine (+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is generally understood in the art (Kyte et al., 1982, J. Mol. Biol. 157: 105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those within ±1 are particularly preferred, and those within ±0.5 are even more particularly preferred.

It is also understood in the art that the substitution of like amino acids can be made effectively based on hydrophilicity, particularly where the biologically functionally equivalent protein or peptide thereby created is intended for use in immunological invention, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those within ±1 are particularly preferred, and those within ±0.5 are even more particularly preferred. One may also identify epitopes from primary amino acid sequences based on hydrophilicity.

Desired amino acid substitutions (whether conservative or non-conservative) can be determined by those skilled in the art at the time such substitutions are desired. For example, amino acid substitutions can be used to identify important residues of the protein, or to increase or decrease the immunogenicity, solubility or stability of the protein.

Methods of this disclosure may use one or more antigens, each of which independently comprises at least 6 contiguous amino acids, at least 10 contiguous amino acids, at least 20 contiguous amino acids, at least 30 contiguous amino acids, at least 50 contiguous amino acids, at least 75 contiguous amino acids, or at least 100 contiguous amino acids. Methods of this disclosure may use one or more antigens, each of which independently comprises an amino acid sequence at least 85% identical, at least 95% identical, at least 97% identical, or at least 99% identical, to at least 10 contiguous amino acids, at least 20 contiguous amino acids, at least 30 contiguous amino acids, at least 50 contiguous amino acids, at least 75 contiguous amino acids, or at least 100 contiguous amino acids. Methods of this disclosure may use one or more antigens, each of which independently comprises at least 6 contiguous amino acids, at least 10 contiguous amino acids, at least 20 contiguous amino acids, at least 30 contiguous amino acids, at least 50 contiguous amino acids, at least 75 contiguous amino acids, or at least 100 contiguous amino acids. Methods of this disclosure may use one or more antigens, each of which independently comprises an amino acid sequence at least 95% identical, at least 97% identical, or at least 99% identical, to 9 to 15 contiguous amino acid residues, wherein the antigen is an MHC I-restricted antigen. Methods of this disclosure may use one or more antigens, each of which independently comprises 9 to 15 contiguous amino acid residues, wherein the antigen is an MHC I-restricted antigen. Methods of this disclosure may use one or more antigens comprising an amino acid sequence at least 95% identical, at least 97% identical, or at least 99% identical, to at least 15 contiguous amino acid residues, wherein the antigen is an MHC II-restricted antigen. Methods of this disclosure may use one or more antigens comprising at least 15 contiguous amino acid residues, wherein the antigen is an MHC II-restricted antigen. Methods of this disclosure may one or more antigens comprising an amino acid sequence at least 95% identical, at least 97% identical, or at least 99% identical, to a peptide consisting of a sequence selected from the group consisting of peptides comprising the amino acid sequence of SEQ ID NOS: 1-90, or any combination thereof. Methods of this disclosure may use one or more antigens consisting of an amino acid sequence at least 95% identical, at least 97% identical, or at least 99% identical, to a sequence selected from the group consisting of peptides comprising the amino acid sequence of SEQ ID NOS: 1-91, or any combination thereof. Methods of this disclosure may use one or more antigens consisting of a sequence selected from the group consisting of peptides comprising the amino acid sequence of SEQ ID NOS: 1-91, or any combination thereof.

Methods of the invention comprise treating an individual for cancer by recruiting a preexisting immune response in the body to the cancer site. In these methods, the individual may be known to have a preexisting immune response to an antigen, prior to initiation of the cancer treatment. The individual may be tested to confirm the presence of a preexisting immune response prior to initiating the cancer treatment. Thus, these methods may include treating cancer in an individual by confirming that the individual has a preexisting immune response to an antigen, wherein the antigen is not present in, or on, the cancer. The PROTEXI is then administered to the individual confirmed to have the preexisting immunity, such that the PROTEXI orchestrates CD4+ T cell help for the activation of tumor-specific CD8+ T cell, thereby treating the cancer.

Any method of confirming that the individual to be treated has a preexisting immune response to an antigen can be used to practice methods of this disclosure. Examples of such methods include identifying in a sample from the individual a B-cell that recognizes a specific antigen, an antibody that recognizes a specific antigen, a T-cell that recognizes a specific antigen, or T-cell activity that is initiated in response to a specific antigen. Any suitable sample from the individual can be used to identify a preexisting immune response. Examples of suitable samples include, but are not limited to, whole blood, serum, plasma, and tissue samples. As used herein, recognition of a specific antigen by a B-cell, T-cell, or an antibody, refers to the ability of such B-cells, T-cells, or antibodies to specifically bind the antigen. Specific binding of an antigen by a B-cell, T-cell, or antibody, means a B-cell, T-cell, or antibody, binds to a specific antigen with an affinity greater than the binding affinity of the same B-cell, T-cell, or antibody, for a molecule unrelated to the antigen. For example, a B-cell, T-cell, or antibody, that recognizes, or is specific for, an antigen from a Coronavirus spike protein, binds the Coronavirus spike protein antigen with an affinity significantly greater than the binding affinity of the same B-cell, T-cell, or antibody, for a protein unrelated to Coronavirus spike protein, such as human albumin. Specific binding between two entities can be scientifically represented by their dissociation constant, which is often less than about 10⁻⁶, less than about 10⁻⁷, or less than about 10⁻⁸ M. The concept of specific binding, and methods of measuring such binding, between molecules, and cells and molecules, are well known to a person of ordinary skill in the art including, but not limited to, enzyme immunoassays (e.g., ELISA), immunoprecipitations, immunoblot assays and other immunoassays as described, for example, in Sambrook et al., supra, and Harlow et al., Antibodies, a Laboratory Manual (Cold Spring Harbor Labs Press, 1988). Such methods are also described in U.S. Pat. No. 7,172,873, which is incorporated herein by reference. Methods of measuring T-cell activation in a sample from an individual are also known to those skilled in the art. Examples of such methods are disclosed in U.S. Patent Publication No. 2003/003485, and in U.S. Pat. No. 5,750,356, both of which are incorporated herein by reference.

Such methods generally comprise contacting a T-cell containing sample from the individual with an antigen, and measuring the sample for activation of T-cells. Methods of measuring T-cell activation are also well known in the art and are also disclosed in Walker, S., et al., Transplant Infectious Disease, 2007:9:165-70; and Kotton, C. N. et al. (2013) Transplantation 96, 333.

Molecules Loaded on to Antigen Presenting Cell

In any of the methods provided in this disclosure, antigens may be pulsed or loaded on to antigen presenting cells such as dendritic cells. However, other types of molecules may also be loaded, such as nucleic acids or recombinant protein. In addition, dendritic cell may be pulsed with exosome or tumor cell lysate originated from the recipient or other patient. The present invention is not limited by the manner of loading a molecule on to the dendritic cell. The loading may occur on any antigen presenting cell such as dendritic cell.

Adjuvant

In any of the methods provided in this disclosure, other agents may be used (i.e., administered), within the practice of the current invention to augment the immune modulatory or recruitment. Such other agents which include, a TLR agonist; intravenous immunoglobulin (IVIG); peptidoglycan isolated from gram positive bacteria; lipoteichoic acid isolated from gram positive bacteria; lipoprotein isolated from gram positive bacteria; lipoarabinomannan isolated from mycobacteria, zymosan isolated from yeast cell wall; polyadenylic-polyuridylic acid; poly (IC); lipopolysaccharide; monophosphoryl lipid A; flagellin; Gardiquimod; Imiquimod; R848; oligonucleosides containing CpG motifs, a CD40 agonist, and 23 S ribosomal RNA In a preferred aspect of these methods, the TLR agonist is poly-IC.

In the following studies, the following MHC-1 and MHC-II restricted peptides are used.

TABLE 1
MHC-I restricted peptides used in the studies

| Mouse | | |
|---|---|---|
| cell lines | Epitopes | MHC-I Peptide |
| F420 | MT4-1 | HRAERPFPEED (SEQ ID NO: 1) |
| | MT4-2 | LTPSFPVSP (SEQ ID NO: 2) |
| | MT4-6 | RVGGHLRLSGQ (SEQ ID NO: 3) |
| | MT4-7 | GVRAALPTPR (SEQ ID NO: 4) |
| | MT4-8 | QAGPVLPTSLE (SEQ ID NO: 5) |
| | B6-Luc2 | LMYRFEEEL (SEQ ID NO: 6) |
| B16F10 | M30-11 | WENVSPELNST (SEQ ID NO: 7) |
| | Trp2 | SVYDFFVWL (SEQ ID NO: 8) |

TABLE 2
MHC-II restricted peptides used in the studies

| Species | Epitopes | MHC-II Peptide | MHC-II |
|---|---|---|---|
| Mouse | OVA₃₂₃ | ISQAVHAAHAEINEAGR (SEQ ID NO: 9) | B6(I-A^{d}) |
| Human | Spike S236 | TRFQTLLALHRSYLT (SEQ ID NO: 10) | HLA-DRB04: |
| | | | 01 |

Below are tables presenting examples of MHC-I and MHC-II restricted peptides that may be used in PROTEXI.

TABLE 3
HLA-DR and DP restricted Spike epitopes selected for PROTEXI production

| | HLA | | | | |
|---|---|---|---|---|---|
| Number | Restrictions | Spike Sequence | Start | End | Length |
| 1 | DRB1_0401, | SFTRGVYYPDKVFRSSVLH (SEQ ID | 31 | 49 | 19 |
| | DRB3_0202, | NO: 11) | | | |
| | DRB3_0101, | | | | |
| | DRB1_0301, | | | | |
| | DRB1_1303 | | | | |
| 2 | DRB3_0202, | RKSNLKPFERDISTEIYQAGSTPC | 457 | 480 | 24 |
| | DRB3_0101, | (SEQ ID NO: 12) | | | |
| | DRB4_0103 | | | | |
| | DPB1_0101 | TQLNRALTGIAVEQDKNT(SEQ ID | 761 | 778 | 18 |
| | | NO: 13) | | | |
| 4 | DRB1_0401 | GFNFSQILPDPSKPSKRSFI(SEQ ID | 799 | 818 | 20 |
| | | NO: 14) | | | |
| 5 | DRB1_0401 | IQDSLSSTASALGKLQDV(SEQ ID | 934 | 951 | 18 |
| | | NO: 15) | | | |
| 6 | DRB1_0102, | GKLQDVVNQNAQALNTLVKQLSSN | 946 | 969 | 24 |
| | DPB1_0101 | (SEQ ID NO: 16) | | | |
| 7 | DRB1_1101, | NAQALNTLVKQLSSNFGAISS (SEQ ID | 955 | 975 | 21 |
| | DRB1_0102 | NO: 17) | | | |
| 8 | DRB1_0301 | TAPAICHDGKAHFPREGV (SEQ ID | 1077 | 1094 | 18 |
| | | NO: 18) | | | |
| 9 | DRB1_0401 | GTHWFVTQRNFYEPQ (SEQ ID NO: 19) | 1099 | 1113 | 15 |
| 10 | DRB1_0301, | EPQIITTDNTFVSGNC (SEQ ID NO: 20) | 1111 | 1126 | 16 |
| | DRB1_0401 | | | | |
| 11 | DRB3_0202, | TTLDSKTQSLLIVNNATNVVIK (SEQ ID | 108 | 129 | 22 |
| | DRB4_0103, | NO: 21) | | | |
| | DPB1_1401 | | | | |
| 12 | DRB_0401, | PFLGVYYHKNNKSW (SEQ ID NO: 22) | 139 | 152 | 14 |
| | DRB_1101 | | | | |
| 13 | DRB_0401, | PTESIVRFPNITNLCPFG (SEQ ID | 322 | 339 | 18 |
| | DRB1_1501 | NO: 23) | | | |
| 14 | DRB1_0701 | IPTNFTISVTTEILPV (SEQ ID NO: 24) | 714 | 729 | 16 |

TABLE 4
Examples of HLA-I restricted NY-ESO-1 epitopes

| CTAs | HLA-I | Epitopes |
|---|---|---|
| ESO 90-100 | A*02:01 | YLAMPFATPM (SEQ ID NO: 25) |
| ESO 158-167 | A*02:01 | LLMWITQCFL (SEQ ID NO: 26) |
| ESO 157-165 | | SLLMWITQC (SEQ ID NO: 27) |
| ESO 157-167 | | SLLMWITQCFL (SEQ ID NO: 28) |
| ESO 60-72 | B*07:02 | APRGPHGGAASGL (SEQ ID NO: 29) |
| ESO 88-96 | B*18:01 | LEFYLAMPF (SEQ ID NO: 30) |
| ESO 96-104 | C*03:04 | FATPMEAEL (SEQ ID NO: 31) |

TABLE 5
Examples of HLA-I restricted MAGE-A1 epitopes

| CTAs | HLA-I | Epitopes |
|---|---|---|
| MAGE-A1 | HLA-A1 | EADPTGHSY (SEQ ID |
| 161-169 | | NO: 32) |
| MAGE-A1 | A*2401 | NYKHCFPEI (SEQ ID NO: 33) |
| MAGE-A1 | A*0201 | KVLEYVIKV (SEQ ID NO: 34) |

TABLE 6
Examples of HLA-I restricted MAGE-A3 epitopes

| CTAs | HLA-I | Epitopes |
|---|---|---|
| MAGE-A3 | HLA-A2 | QLVFGIELMEV (SEQ ID NO: 35) |
| 158-169 | | |
| MAGE-A3 | HLA-A1 | EVDPIGHLY (SEQ ID NO: 36) |
| 167-175 | | |
| MAGE-A3 | HLA-A2 | IMPKAGLLIIV (SEQ ID NO: 37) |
| 194-205 | | |
| MAGE-A3 | HLA-A24 | IMPKAGLLI (SEQ ID NO: 38) |
| 194-203 | | |
| MAGE-A3 | HLA-A2 | FLWGPRALV (SEQ ID NO: 39) |
| 271-279 | | |
| MAGE-A3 | HLA-A2 | KVAELVHFL (SEQ ID NO: 40) |
| 112-120 | | |

TABLE 7
Examples of HLA-I restricted PRAME epitopes

| CTAs | HLA-I | Epitopes |
|---|---|---|
| PRAME 435-443 | A*02:01 | NLTHVLYPV (SEQ ID NO: 41) |
| PRAME 301-309 | A24 | LYVDSLFFL (SEQ ID NO: 42) |
| PRAME 100-108 | A*02:01 | VLDGLDVLL (SEQ ID NO: 43) |
| PRAME 142-151 | A*02:01 | SLYSFPEPEA (SEQ ID |
| | | NO: 44) |
| PRAME 300-309 | A*02:01 | ALYVDSLFFL (SEQ ID |
| | | NO: 45) |
| PRAME 425-433 | A*02:01 | SLLQHLIGL (SEQ ID NO: 46) |

Overcoming the Limitations of Currently Available MHC-II NeoAg Screening Methods

Genomic mutations are a major driver of carcinogenesis and malignant cancer progression. Since the first study reported that many non-synonymous mutants are immunogenic (NeoAgs) and may confer anti-tumor vaccine activity[14], personalized tumor vaccine approaches have evolved rapidly during last decade and generated promising results in clinical evaluations. Identification of highly immunogenic NeoAgs through MHC-I and MHC-II-restricted peptide screening algorithms has the potential to optimize efforts to promote immune-mediated tumor rejection. While CD4+ T helper cells play a key role, MHC-II NeoAg screening algorithms are limited in their capacity to predict highly immunogenic NeoAgs due to the less stringent nature of MHC-II compared to MHC-I molecules. Individualized NeoAgs are used in the form of peptide or mRNA encoding long peptides (15-30mers) encompassing CD8+ T cell epitopes identified by MHC-I epitope prediction [15] simply because of the inaccuracy of MHC-II epitope prediction. Without screening or applying presumably less immunogenic MHC-II NeoAgs, the novel anti-tumor vaccine approach with PROTEXI (Celloram Inc.), an autologous DC-based tumor vaccine platform co-presenting clinically proven MHC-II PAA epitopes coupled with MHC-I NeoAgs, provides a unique, unprecedented solution for a robust CD4+ helper T cell activation. More significantly, the PROTEXI vaccine consistently promotes a tumor microenvironment in which CD4+ T cells support tumor-specific CTL activation primarily by cytokine secretion via their close proximity.

A Vaccine Designed to Promote Epitope Spreading

Epitope spreading is characterized by diversified T cell expansion that is not related to the originally vaccinated epitopes. In the clinical setting, epitope spreading is an important mechanism leading to durable protection in response to immunotherapies including immune checkpoint blockade, CAR-T, ACT, and NeoVax treatment[7, 15]. Interestingly, DCs play a central role in endogenous antigen uptake and cross-presentation for the induction of epitope spreading. As such, the PROTEXI platform is in a unique position that recapitulates the epitope spreading mechanism by simultaneously presenting MHC-II PAA epitopes and MHC-I NeoAgs, thereby the proposed method of induction of epitope spreading will lead to PAA-specific CD4+ T cell-mediated CD8+ T cell expansion, regardless of CD4+ T antigen specificity. Currently, there are no clinical strategies designed to specifically induce epitope spreading.

Advantages of PROTEXI Over Other Existing Vaccine Platforms

The PROTEXI platform advantageously possesses the characteristics of high immunogenicity [16], low potential toxicity, and no limitations to convey any HLA-restricted epitopes relative to other vaccine platforms. Because PROTEXI is a cell-based product, cost will be higher when compared to mRNA, DNA, peptide or viral vaccines. While mRNA platforms are viewed as more readily scalable (as exemplified in SARS-CoV-2 vaccination), DC platforms like PROTEXI are also feasible and affordable for use in personalized cancer vaccines. More importantly, PROTEXI is a unique platform which directly delivers the proposed epitope spreading mechanism to cancer patients for the induction of optimal anti-tumor immune responses.

The autologous personalized DC designed to promote epitope spreading would not only be impactful for patients with OS, but also for other cancers that have relatively low tumor mutational burden (TMB) because it can potentiate the diversification or promotion of the subdominant T cells into immuno-dominant CTL. Given the safety of the COVID-19 vaccine, utilizing Spike protein/peptides in our PROTEXI platform would be a safe approach to boost anti-tumor immunity and would be highly feasible for clinical application in cancer patients. In addition, combination with immune modulators including immune checkpoint inhibitors and a TGF-beta signaling inhibitor (Vactosertib) maximizes PROTEXI efficacy, by blocking the immune suppressive signals in the tumor microenvironment. Therefore, the PROTEXI platform would transform the personalized DC NeoAg-based vaccine approaches in future clinical trials, advancing a new, state-of-the-art immunotherapy not only for AYA osteosarcoma patients but also for other cancer patients.

An inhibitor of transforming growth factor (TGF)-beta receptor type 1 (TGFBR1) may be used in conjunction with the PROTEXI of the present invention, such as Vactosertib. Vactosertib is a small molecule and an orally bioavailable inhibitor of the serine/threonine kinase, transforming growth factor (TGF)-beta receptor type 1 (TGFBR1), also known as activin receptor-like kinase 5 (ALK5), with potential antineoplastic activity[17]. Upon oral administration, Vactosertib inhibits the activity of TGFBR1 and prevents TGF-beta/TGFBR1-mediated signaling. This suppresses tumor growth in TGFBR1-overexpressing tumor cells. TGFBR1, which is overexpressed in a variety of tumor cell types, plays a key role in tumor cell proliferation. Expression of TGF-beta promotes tumor cell proliferation, enhances the migration of tumor cells and suppresses the response of the host immune system to tumor cells. Anti-cancer synergistic activity is contemplated.

Personalized Neoantigens

First, the tumor tissue and blood are delivered to the sequencing laboratory for analyzing the status of the tumor and normal, respectively. DNA in the patient's blood is utilized for the comparison to the tumor.

Given tumor and blood, whole-exome sequencing (WES) is applied to screen the somatic variants, which are ‘non-self’ mutation including point mutation, short insertional/deletional mutation or structural variant only in the tumor. Next, the expression of ‘non-self’ somatic variants into RNA is then examined by using RNA-Sequencing (RNA-Seq). The unique HLA alleles for each patient can be determined based on either WES or RNA-Seq.

Given that ‘non-self’ somatic variants are expressed into RNA, the binding affinity and immunogenicity of variants are predicted by using computer-based neoantigen screening algorithm. Through this process, neoantigens, which are suggested to be ‘non-self’ somatic variants found only in the tumor, are prioritized based on potential to bind with the patient's HLA and to be most likely immunogenic to patient's immune system.

Public Neoantigens

Neoantigen for autologous dendritic cells may be varied, as they are specific to the individual. However, publicly known neoantigens may also be used to load the dendritic cell to obtain the PROTEXI system. Without limitation, some of the publicly known neoantigens are listed in Tables 4 to 7. The contents of the review article Durgeau et al., “Recent Advances in Targeting CD8 T-Cell Immunity for More Effective Cancer Immunotherapy”, Front. Immunol., 22 Jan. 2018|doi.org/10.3389/fimmu.2018.00014 is incorporated by reference herein for disclosure with respect to the various public neoantigens that are available as well as discussion of advances in CD8 T-cell targeted cancer therapy.

CD4 T-Cell Activating Compounds

MHC-II peptide complex for helper T-cell activation loaded on to dendritic cells is a feature of the present invention. Without limitation some of the candidate CD4 T-cell activating peptides are listed in Table 3. The contents of the review article Parker et al., “Mapping the SARS-CoV-2 spike glycoprotein-derived peptidome presented by HLA class II on dendritic cells”, Cell Reports, 2021, 35, 109179 is incorporated by reference herein for disclosure with respect to the various CD4 T-cell activating peptides or agents that are known in the art.

Other MHC-II epitopes may be as indicated in Table 8 below, which can be found in Liang et al., “Population-Predicted MHC Class II Epitope Presentation of SARS-CoV-2 Structural Proteins Correlates to the Case Fatality Rates of COVID-19 in Different Countries”, Int. J. of Mol. Sci., 2021, 22, 2630, which is incorporated by reference herein for disclosure with respect to the various CD4 T cell activating peptides or agents that are known in the art.

TABLE 8
MHC class II epitopes and their recognizing alleles used for the population coverage
analysis.

| | | |
|---|---|---|
| Spike protein | AAEIRASANLAATKM (SEQ ID | HLA-DRB1*01:01 |
| | NO: 47) | HLA-DRB1*13:02 |
| | | HLA-DRB3*02:02 |
| | | HLA-DRB1*04:01 |
| | | HLA-DQA1*05:01/DQB1*03:01 |
| | | HLA-DRB1*07:01 |
| | | HLA-DRB1*07:01 |
| | | HLA-DRB1*09:01 |
| | AQKFNGLTVLPPLLT (SEQ ID | HLA-DRB1*01:01 |
| | NO: 48) | HLA-DRB1*04:05 |
| | | HLA-DRB1*04:01 |
| | | HLA-DRB1*09:01 |
| | | HLA-DRB1*07:01 |
| | ATRFASVYAWNRKRI (SEQ ID | HLA-DRB5*01:01 |
| | NO: 49) | HLA-DRB1*01:01 |
| | | HLA-DRB1*07:01 |
| | | HLA-DRB1*09:01 |
| | | HLA-DRB1*11:01 |
| | | HLA-DRB1*15:01 |
| | DEMIAQYTSALLAGT (SEQ ID | HLA-DRB1*01:01 |
| | NO: 50) | HLA-DRB1*15:01 |
| | DLFLPFFSNVTWFHA (SEQ ID | HLA-DRB1*01:01 |
| | NO: 51) | HLA-DRB1*15:01 |
| | | HLA-DRB1*07:01 |
| | | HLA-DRB1*04:05 |
| | | HLA-DPA1*01:03/DPB1*02:01 |
| | | HLA-DRB1*04:01 |
| | | HLA-DRB1*09:01 |
| | | HLA-DRB3*02:02 |
| | EDLLFNKVTLADAGF (SEQ ID | HLA-DRB1*13:02 |
| | NO: 52) | HLA-DRB1*01:01 |
| | | HLA-DRB1*13:02 |
| | FFSNVTWFHAIHVSG (SEQ ID | HLA-DRB1*07:01 |
| | NO: 53) | HLA-DRB1*01:01 |
| | | HLA-DRB1*15:01 |
| | | HLA-DRB1*09:01 |
| | FGEVFNATRFASVYA (SEQ ID | HLA-DPA1*01:03/DPB1*02:01 |
| | NO: 54) | HLA-DRB1*07:01 |
| | | HLA-PA1*02:01/DPB1*01:01 |
| | | HLA-DRB1*01:01 |
| | | HLA-DRB1*09:01 |
| | | HLA-DPA1*03:01/DPB1*04:02 |
| | | HLA-DRB1*11:01 |
| | FSTFKCYGVSPTKLN (SEQ ID | HLA-DRB1*01:01 |
| | NO: 55) | HLA-DRB1*07:01 |
| | GSNVFQTRAGCLIGA (SEQ ID | HLA-DRB1*01:01 |
| | NO: 56) | HLA-DRB1*07:01 |
| | GWTFGAGAALQIPFA (SEQ ID | HLA-DQA1*05:01/DQB1*03:01 |
| | NO: 57) | HLA-DRB1*09:01 |
| | | HLA-DRB1*07:01 |
| | IIAYTMSLGAENSVA (SEQ ID | HLA-DRB1*01:01 |
| | NO: 58) | HLA-DRB1*09:01 |
| | | HLA-DRB1*07:01 |
| | | HLA-DRB1*04:01 |
| | | HLA-DRB5*01:01 |
| | | HLA-DRB1*04:05 |
| | ITRFQTLLALHRSYL (SEQ ID | HLA-DRB5*01:01 |
| | NO: 59) | HLA-DRB1*01:01 |
| | | HLA-DRB1*11:01 |
| | | HLA-DRB1*15:01 |
| | | HLA-DRB1*04:05 |
| | | HLA-DRB4*01:01 |
| | | HLA-DRB1*07:01 |
| | | HLA-DRB1*04:01 |
| | | HLA-DRB1*09:01 |
| | KRSFIEDLLFNKVTL (SEQ ID | HLA-DPA1*01:03/DPB1*02:01 |
| | NO: 60) | HLA-DPA1*02:01/DPB1*01:01 |
| | | HLA-DPA1*03:01/DPB1*04:02 |
| | LQIPFAMQMAYRFNG (SEQ ID | HLA-DRB1*01:01 |
| | NO: 61) | HLA-DRB5*01:01 |
| | | HLA-DRB1*07:01 |
| | | HLA-DRB1*15:01 |
| | | HLA-DRB1*11:01 |
| | | HLA-DRB1*09:01 |
| | | HLA-DRB4*01:01 |
| | | HLA-DRB1*01:01 |
| | NCTFEYVSQPFLMDL (SEQ id | HLA-DPA1*01:03/DPB1*02:01 |
| | NO: 62) | HLA-DPA1*02:01/DPB1*01:01 |
| | | HLA-DRB1*01:01 |
| | | HLA-DPA1*03:01/DPB1*04:02 |
| | | HLA-DRB1*07:01 |
| | NQKLIANQFNSAIGK (SEQ ID | HLA-DRB1*13:02 |
| | NO: 63) | HLA-DRB3*02:02 |
| | | HLA-DRB1*01:01 |
| | NSVAYSNNSIAIPTN (SEQ ID | HLA-DRB1*13:02 |
| | NO: 64) | HLA-DRB3*02:02 |
| | NYNYLYRLFRKSNLK (SEQ ID | HLA-DRB5*01:01 |
| | NO: 65) | HLA-DRB1*15:01 |
| | | HLA-DRB1*01:01 |
| | PIGINITRFQTLLAL (SEQ ID | HLA-DRB1*15:01 |
| | NO: 66) | HLA-DRB1*01:01 |
| | | HLA-DPA1*01:03/DPB1*02:01 |
| | | HLA-DRB1*13:02 |
| | | HLA-DRB4*01:01 |
| | PINLVRDLPQGFSAL (SEQ ID | HLA-DRB1*03:01 |
| | NO: 67) | HLA-DRB3*01:01 |
| | | HLA-DRB1*13:02 |
| | PTNFTISVTTEILPV (SEQ ID | HLA-DRB1*07:01 |
| | NO: 68) | HLA-DRB1*01:01 |
| | | HLA-DRB1*09:01 |
| | QMAYRFNGIGVTQNV (SEQ ID | HLA-DRB1*01:01 |
| | NO: 69) | HLA-DRB3*02:02 |
| | QPYRVVVLSFELLHA | HLA-DPA1*01:03/DPB1*02:01 |
| | (SEQ ID NO: 70) | HLA-DPA1*02:01/DPB1*01:01 |
| | | HLA-DPA1*03:01/DPB1*04:02 |
| | QQLIRAAEIRASANL (SEQ ID | HLA-DRB1*01:01 |
| | NO: 71) | HLA-DRB4*01:01 |
| | | HLA-DQA1*05:01 /DQB 1*03:01 |
| | | HLA-DQA1*01:02/DQB 1*06:02 |
| | | HLA-DRB1*08:02 |
| | | HLA-DRB5*01:01 |
| | QSLLIVNNATNVVIK (SEQ ID | HLA-DRB1*13:02 |
| | NO: 72) | HLA-DRB3*02:02 |
| | | HLA-DRB1*01:01 |
| | | HLA-DRB1*04:01 |
| | | HLA-DRB1*07:01 |
| | QTYVTQQLIRAAEIR (SEQ ID | HLA-DRB1*01:01 |
| | NO: 73) | HLA-DRB4*01:01 |
| | REGVFVSNGTHWFVT (SEQ ID | HLA-DRB1*13:02 |
| | NO: 74) | HLA-DRB3*02:02 |
| | | HLA-DRB1*07:01 |
| | | HLA-DRB1*01:01 |
| | | HLA-DRB1*09:01 |
| | RGVYYPDKVFRSSVL (SEQ ID | HLA-DRB3*01:01 |
| | NO: 75) | HLA-DRB1*01:01 |
| | SEFRVYSSANNCTFE (SEQ ID | HLA-DRB1*01:01 |
| | NO: 76) | HLA-DRB1*09:01 |
| | SFVIRGDEVRQIAPG (SEQ ID | HLA-DRB1*03:01 |
| | NO: 77) | HLA-DRB3*01:01 |
| | SSGWTAGAAAYYVGY (SEQ ID | HLA-DQA1*05:01/DQB1*03:01 |
| | NO: 78) | HLA-DRB1*09:01 |
| | | HLA-DRB1*01:01 |
| | | HLA-DQA1*01:02/DQB1*06:02 |
| | TGRLQSLQTYVTQQL (SEQ ID | HLA-DRB1*01:01 |
| | NO: 79) | HLA-DRB1*15:01 |
| | TLLALHRSYLTPGDS (SEQ ID | HLA-DRB1*01:01 |
| | NO: 80) | HLA-DRB1*15:01 |
| | TSNFRVQPTESIVRF (SEQ ID | HLA-DRB1*01:01 |
| | NO: 81) | HLA-DRB1*04:01 |
| | | HLA-DRB1*07:01 |
| | | HLA-DRB1*04:05 |
| | | HLA-DRB1*13:02 |
| | | HLA-DRB1*09:01 |
| | TVEKGIYQTSNFRVO (SEQ ID | HLA-DRB1*07:01 |
| | NO: 82) | HLA-DRB1*13:02 |
| | | HLA-DRB1*01:01 |
| | TWRVYSTGSNVFQ.TR (SEQ ID | HLA-DRB1*07:01 |
| | NO: 83) | HLA-DRB1*01:01 |
| | | HLA-DRB1*09:01 |
| | VKQLSSNFGAISSVL (SEQ ID | HLA-DRB1*13:02 |
| | NO: 84) | HLA-DRB1*01:01 |
| | | HLA-DRB3*02:02 |
| | | HLA-DQA1*05:01/DQB1*03:01 |
| | VLSFELLHAPATVCG (SEQ ID | HLA-DRB1*01:01 |
| | NO: 85) | HLA-DRB1*09:01 |
| | VNFNFNGLTGTGVLT(SEQ ID | HLA-DRB1*01:01 |
| | NO: 86) | HLA-DRB1*09:01 |
| | | HLA-DRB1*07:01 |
| | YFKIYSKHTPINLVR (SEQ ID | HLA-DRB1*01:01 |
| | NO: 87) | HLA-DRB1*07:01 |
| | | HLA-DRB5*01:01 |
| | | HLA-DRB1*11:01 |
| | | HLA-DRB1*09:01 |
| | YRLFRKSNLKPFERD (SEQ ID | HLA-DRB5*01:01 |
| | NO: 88) | HLA-DRB1*11:01 |
| | YSVLYNSASFSTFKC (SEQ ID | HLA-DRB1*01:01 |
| | NO: 89) | HLA-DRB1*13:02 |
| | | HLA-DRB3*02:02 |
| | | HLA-DRB1*07:01 |
| | YTSALLAGTITSGWT (SEQ ID | HLA-DQA1*05:01/DQB1*03:01 |
| | NO: 90) | HLA-DRB1*01:01 |
| | YYVGYLQPRTFLLKY (SEQ ID | HLA-DRB1*01:01 |
| | NO: 91) | HLA-DRB5*01:01 |
| | | HLA-DRB1*07:01 |
| | | HLA-DRB1*15:01 |
| | | HLA-DRB1*09:01 |
| | | HLA-DPA1*01:03/DPB1*02:01 |

The medicament of the invention may also include one or more adjuvants. Adjuvants are substances that non-specifically enhance or potentiate the immune response (e.g., immune responses mediated by CD8-positive T cells and helper-T (TH) cells to an antigen and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLRS ligands derived from flagellin, FLT3 ligand, GM-CSF, 1030, 1031, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously. Also, cytokines may be used. Several cytokines have been directly linked to influencing dendritic cell migration to lymphoid tissues (e.g., TNF-α), accelerating the maturation of dendritic cells into efficient antigen-presenting cells for T-lymphocytes).

Effective dosages and schedules for administering the loaded dendritic cells may be determined empirically, and making such determinations is within the skill in the art.

To recap, the present application discloses for the first time the following:

Pre-vaccination with DC-OVA323 (preVax-OVA323) set the stage of PROTEXI-mediated anti-tumor immunity via OVA323-specific CD4T cell expansion. Thus, usefulness of pre-existing enhanced CD4+ T helper cells in treating cancer in humans is indicated by monitoring the effects of administering DC-OVA323 as pre-vaccination in mouse model. Administering preVax(OVA323) in mouse model mimics the pre-existing enhanced immune condition in humans such as may have been induced intentionally or unintentionally.

PROTEXI+preVax(OVA323) was successful in harnessing nontumor-specific CD4 T cells and NeoAg-specific CD8 T cells by which significantly enhanced immune-mediated tumor rejection in F420 osteosarcoma model was obtained with increase of CD3+T infiltration (TIL) in the tumor.

PROTEXI+preVax(OVA323) vaccination further enhanced B16F10 tumor rejection over DC pulsed with TAAs (M30-11, Trp2).

To see the direct effect of CD4+T cell, OTII CD4+T(OVA323TCR+) cells were directly injected with PROTEXI loaded with TAA and OVA323 peptides in B16F10 melanoma model. PROTEXI+CD4+T empowered immune-mediated tumor rejection by leveraging non-tumor specific CD4+ helper T cells that is not only effectively driving the expansion of TAA-specific CTL, but also inducing epitope-spreading to unvaccinated Luc2-specific T cells.

PROTEXI+CD4+T cells converted immune-cold to immune-hot tumor via the vigorous recruitment of TIL (CD4+, CD8+, CD11c+ cells).

To demonstrate the pivotal role of CD4+T helper for PROTEXI, the mice in B16F10 melanoma model were administered with anti-CD4 antibody for CD4+T cell depletion or IgG antibody prior to PreVax(OVA323)+PROTEXI vaccination. PROTEXI-induced CD8+T cell directed response to melanoma tumor antigens (Trp2) is abrogated by depletion of CD4+ T cells.

In the absence of CD4+T helper cells, PROTEXI-induced therapeutic efficacy was almost completely vanished due to the lack of polyfunctional CD8T cells (IFN-γ+, TNF-α+, IL-2+) and effector memory T cell population that are important for durable anti-tumor responses.

To test the effect of Spike epitope for PROTEXI in humans, humanized mice expressing HLA-DRB*0401(DR4) were employed. Administration of PROTEXI showed F420 osteosarcoma rejection in humanized DR4 mice in the presence of DRB*0401-restricted Spike (S236) epitope as potent as OVA323 CD4T epitope, suggesting that PROTEXI may exploit Spike CD4 T epitopes by leveraging the global SARS-CoV-2 immunity to strengthen anti-tumor immunity of TAA/TSA, especially important for patient with immune-cold and low TMB tumors including sarcomas.

Tables 3 to 7 above are examples of immune-determinants restricted for HLA-I derived from cancer/testis antigens (CTAs) (e.g., NY-ESO-1, MAGE-A1/3, PRAME) and HLA-II derived from Spike protein of SARS-CoV-2, which are candidates loadable to PROTEXI.

The HLA-I restricted-epitopes could be extended to NeoAgs, CTAs, TAAs, and such.

The HLA-II restricted-epitopes could be extended to Spike (SARS-CoV-2), HA(Influenza), CMV, HPV, EBV, HBV, HTLV, and such.

Pharmaceutical Compositions

For administration to a subject, the compositions described herein can be provided in pharmaceutically acceptable compositions. These pharmaceutically acceptable compositions comprise PROTEXI and optionally an antigen that functions to enhance immune response of T helper cells.

As used here, the term “pharmaceutically acceptable” refers to those materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid diluent, excipient.

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

As used herein, the term “co-administer” refers to administration of two or more therapies or two or more therapeutic agents (e.g., PROTEXI and additional anti-cancer therapies) within a 24 hour period of each other, for example, as part of a clinical treatment regimen. In other embodiments, “co-administer” refers to administration within 12 hours, within 6 hours, within 5 hours, within 4 hours, within 3 hours, within 2 hours, within 1 hour, within 45, within 30 minutes, within 20, within 15 minutes, within 10 minutes, or within 5 minutes of each other. In other embodiments, “co-administer” refers to administration at the same time, either as part of a single formulation or as multiple formulations that are administered by the same or different routes. For example, when the PROTEXI and the additional anti-cancer therapy are administered in different pharmaceutical compositions or at different times, routes of administration can be same or different.

“Contacting” as used here with reference to contacting a cell with an agent (e.g., a compound disclosed herein) refers to any method that is suitable for placing the agent on, in or adjacent to a target cell. For example, when the cells are in vitro, contact the cells with the agent can comprise adding the agent to culture medium containing the cells. For example, when the cells are in vivo, contacting the cells with the agent can comprise administering the agent to the subject.

As used herein, the term “administering” refers to the placement of an agent or a composition as disclosed herein into a subject by a method or route which results in at least partial localization of the agents or composition at a desired site such that a desired effect is produced. Routes of administration suitable for the methods of the invention include both local and systemic administration. Generally, local administration results in more of the composition being delivered to a specific location as compared to the entire body of the subject, whereas, systemic administration results in delivery to essentially the entire body of the subject.

Combination Therapies

In exemplary embodiments, existing treatments for cancer (for use in combination with PROTEXI as described herein) include but are not limited to chemotherapy, radiation therapy, hormonal therapy, surgery, or combinations thereof.

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In a particular embodiment, Vactosertib may be combinationally administered for conditioning hostile tumor microenvironment or DNMT inhibitors (e.g. Azacitidine, Decitabine) for enhancement of IFN-γ T cells and enhanced expression of CTA genes in the tumor. The combinational administration may occur such that the components are administered in mixed state or from separate containers.

In various embodiments, radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or tele-therapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1—Materials and Experimental Methods

We utilize OT-II transgenic mouse [18] that has a C57BL/6 background and expresses the OVA323-339 peptide-responsive CD4 T cell receptor, accounting for ˜80% of total CD4+ T cell population. To assess the immune response of PROTEXI-Spike(S) epitope, we employ the HLA-DR4 mice [19], a murine MHC-II deficient HLA-DRB1*0401 transgenic mice (C57BL/6 background), for the induction of CD4 T cell activity after vaccination of HLA-DRB1*0401 restricted Spike epitopes. For syngeneic mouse models utilizing the OT-II, C57BL/6, and HLA-DR4 mice, we administer B16F10-luciferase cells or metastatic F420-luciferase OS cells derived from a genetically engineered C57BL/6 mouse (Col2.3—Cre/p53R172H) [20].

### Example 1.1—Bone Marrow Derived Dendritic Cell Differentiation and PROTEXI Production

The bone marrow-derived dendritic cells (BMDCs) were differentiated in the media containing GM-CSF (20 ng/ml) and IL-2 (10 ng/ml) for 8 days and matured by treating a maturation cocktail, IFN-γ (1000 U/ml), poly(I:C) (20 μg/ml), TNF-α (5 ng/ml), and IL-1β (25 ng/ml) for an additional day. The immature and mature BMDCs were characterized with expression of surface markers (CD11c, CD40, CD80, CD86, MHC-II).

DC_{TAA} is produced by pulsing the BMDCs with peptide epitopes (5 μg/ml of Trp2 and M30-11) followed by maturation cocktail treatment.

DC_{OVA323} is produced by pulsing the BMDCs with peptide epitope (5 μg/ml of OVA323) followed by maturation cocktail treatment.

PROTEXI is produced by pulsing the BMDCs with peptide epitopes (5 μg/ml of OVA323, Trp2, M30-11) followed by maturation cocktail treatment.

Pre-Vaccination is with DC_{OVA323}

Vaccination is with PROTEXI

Pre-vaccination (Pre-Vax) is conducted by one-time injection of DC pulsed OVA323 (1×10⁶/inj, SQ) at least −7 d.

PROTEXI or DC_{TAA} vaccination is conducted by one-time or multiple injections (1×10⁶/inj, SQ) from d0 of tumor injection.

### Example 1.2—Experimental Cohorts

1) Control, 2) PROTEXI, 3) PreVax+PROTEXI

1) Control, 2) OTII CD4T+DC_{TAA}, 3) OTII CD4T+ DC_{OVA323}, 4) OTII CD4T+PROTEXI

1) Control, 2) PreVax+PROTEXI (IgG), 3) PreVax+PROTEXI (αCD4)

Between day 21 and 28 as tumor size is reaching to >150 mm, the mice are sacrificed for (a) immunotyping (CD4/CD8, IFN-γ, CD25/FOXP3(Treg), CD44/CD62L(Tmem)), (b) cytokines (IFN-γ, IL-2, TGF-β) in the peripheral blood, and (c) NeoAg specific T cell immunity of spleen, draining lymph nodes, and tumor infiltrating leukocytes(TIL) demonstrated by either FACS analysis, ELISA, or CD4/CD8 IFN-γ ELISPOT assay. The peptide dose-dependent ELISPOT assay are conducted for OVA323 and NeoAgs. Excised tumors are subjected to (d) immunostaining for quantification of TIL(CD3+), DC(CD11c+), and macrophages (F4/80).

We collect data for (a) survival and tumor burden, (b) expanded immune cells, (c) the magnitude of epitope-specific T cell activation, (d) cytokines, and (e) tumor tissue staining for CD3+, CD4+, CD8+ TIL, and DC(CD11c).

### Example 2—Animal Models

### Example 2.1—Use of Vertebrate Animal Models

We purchase C57Bl/6 mice (Jackson Laboratory; Stock #000664), OT-II mice (Jackson Laboratory; Stock #004194), and Abb-knockout/HLA-DR4 transgenic mice (Taconic, Stock #4149) for DC cell culture preparations and the subsequent experiments. All these mice are bred and maintained in a manner so that we can have enough number of age and sex-matched mice with their respective controls necessary for this study. All of C57BL/6 and HLA-DR4 mice are pre-vaccinated with either OVA323-339 and/or Spike peptides (S236) prior to F420 OS implantation and therapeutic vaccination.

### Example 3—PROTEXI-Mediated Immune Rejection Against Mouse Model for Osteosarcoma

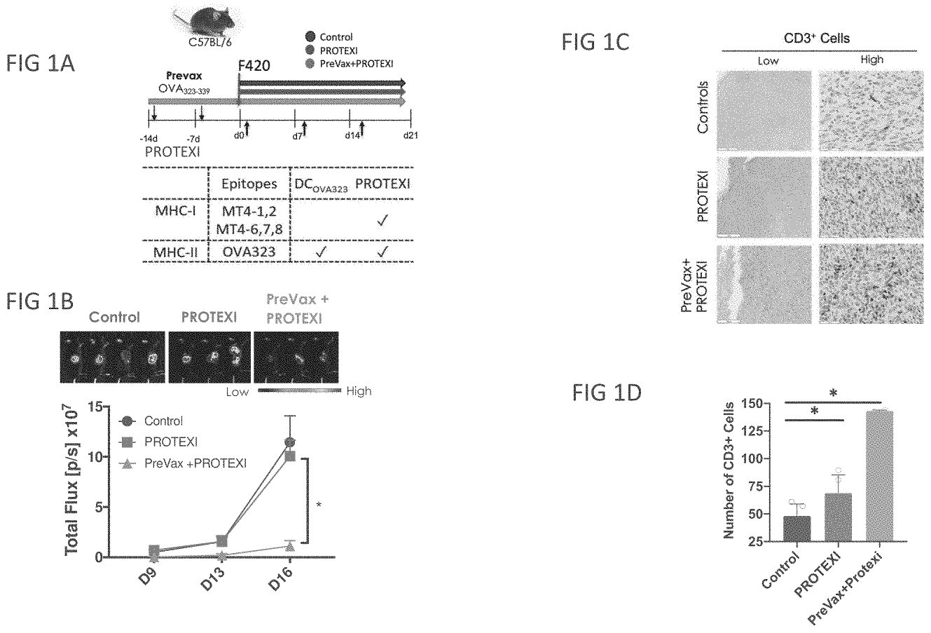

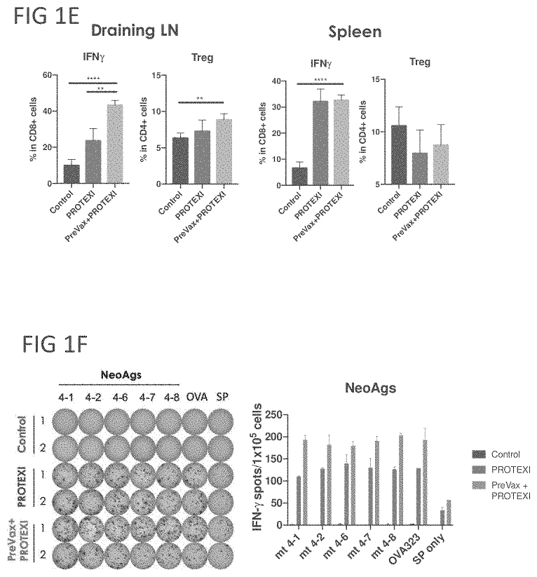

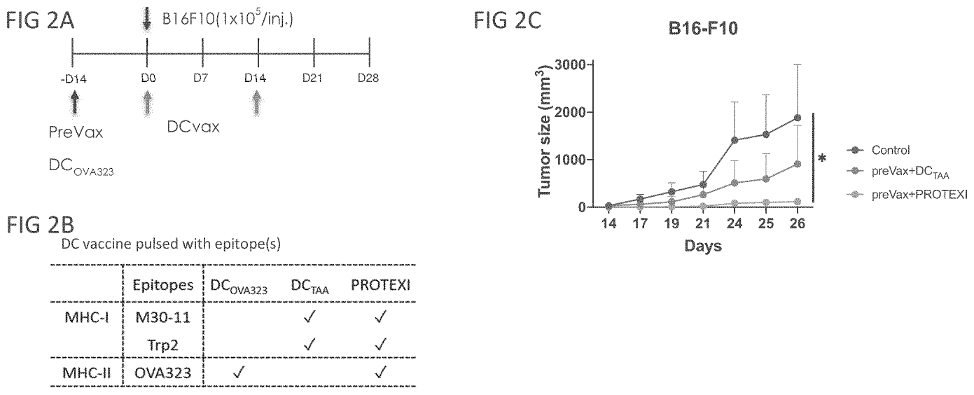

To test the potential of PROTEXI-mediated immune rejection, syngeneic tumor models with F420 (FIG. 1), a murine osteosarcoma (C57BL/6) or B16F10 melanoma (FIG. 2) were employed. The F420 and B16F10 have characteristics of immune-cold tumor.

As a proof-of-principle study, tumor rejection potentials were compared in three treatment groups, control, PROTEXI, and PreVax+PROTEXI, where pre-vaccination (PreVax) conducted 2-times (OVA₃₂₃-pulsed DC, 1×10⁶/injection). To mimic the early-stage of tumor development, the therapeutic vaccinations were given at d0, d7, and d14 following the F420-luc tumor challenge at d0. F420 tumor burdens of the cohorts were monitored by BLI analysis over time followed by CD3+TIL, Flow cytometry, and ELISPOT (IFN-γ) assay. PROTEXI enhanced F420 tumor rejection with pre-vaccination of nontumor-related CD4 epitope, OVA323. PROTEXI increased 2 or 3-fold of CD3+ TIL in F420 tumor group with PreVax+PROTEXI. PROTEXI with DC_{OVA323} pre-vaccination significantly enhanced CD8+IFN-γ+ cytotoxic T cells in draining LN and SP. ELISPOT (IFN-γ) assay depicted the increase of neoantigen (MT4-1, 4-2, 4-6, 4-7, 4-8) and OVA323-reactive T cells in PreVax-PROTEXI group. Therefore, harnessing nontumor-specific CD4 T cell and NeoAg-specific CD8 T cells through PreVax-PROTEXI significantly enhanced immune-mediated tumor rejection of F420 osteosarcoma. See FIG. 1.

### Example 4—Effect of PROTEXI Against Mouse Model for Melanoma

Since PROTEXI vaccination platform has shown an exciting result for immune-mediated tumor rejection in F420 osteosarcoma model, to further explore therapeutic potential of PROTEXI, we employed B16F10 melanoma model. The treatment groups are divided as 1) Control, 2) PROTEXI, and 3) PreVax+PROTEXI. The mice in PreVax group were vaccinated (1×10⁶/inj. SQ) with DC pulsed OVA₃₂₃ immunopeptide at −d14. The B16F10 cell (1×10⁵/inj. SQ) were injected to all of treatment groups followed by two-time injection of PROTEXI (1×10⁶/inj. SQ) pulsed with OVA323, and CD8-specific epitopes (Trp2, and M30-11). The tumor size were measured by caliper. PROTEXI vaccination further enhanced B16F10 tumor rejection over DC pulsed with TAAs (M30-11, Trp2). See FIG. 2.

### Example 5—OTII-CD4T Cell and PROTEXI Against Mouse Model for Melanoma

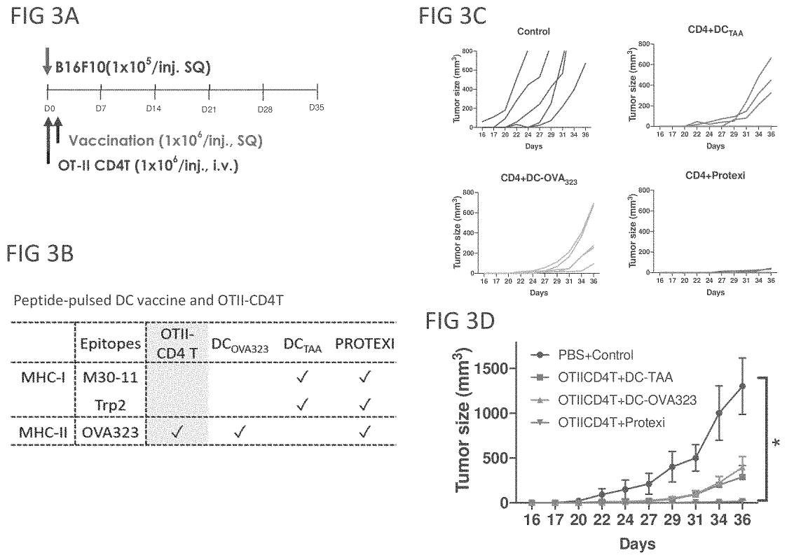

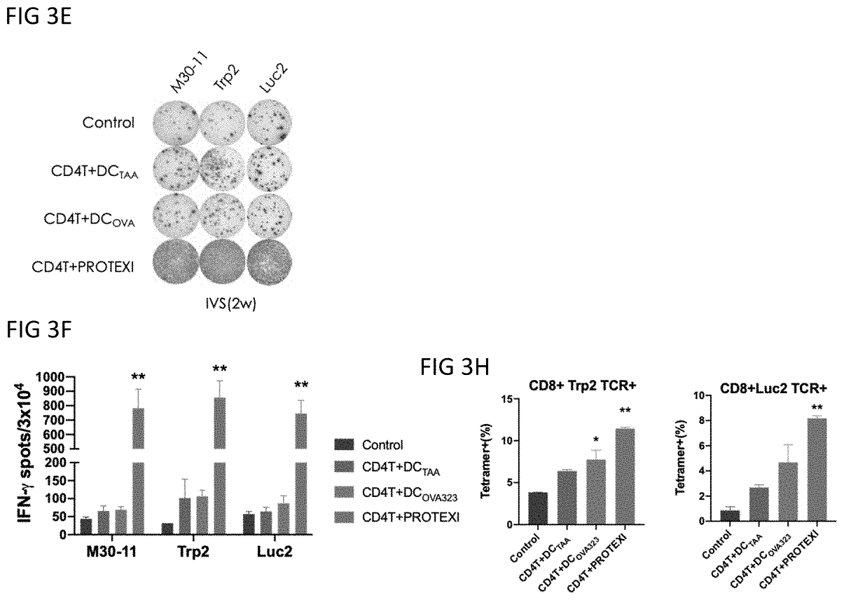

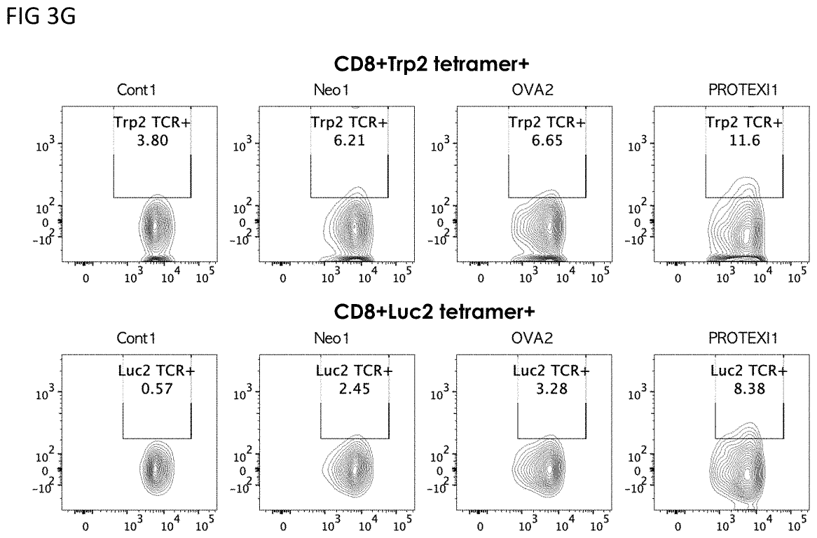

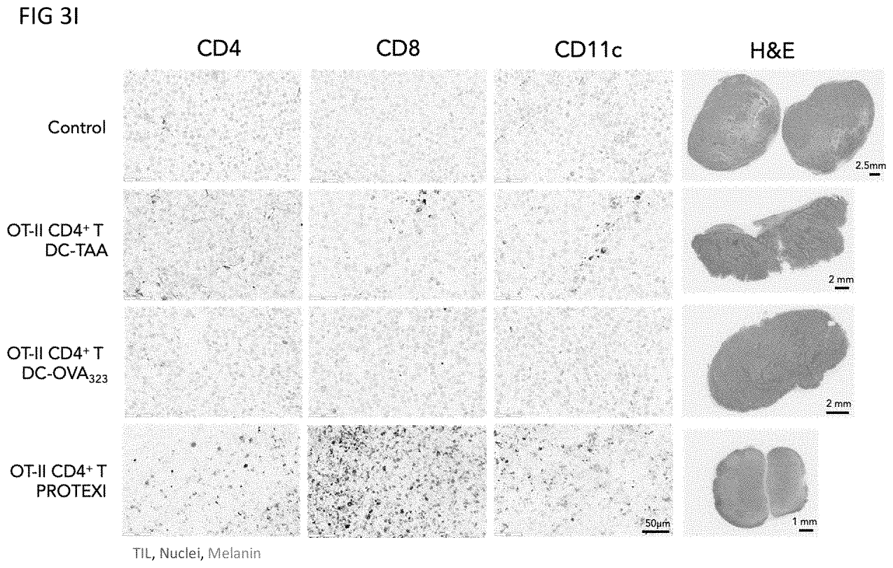

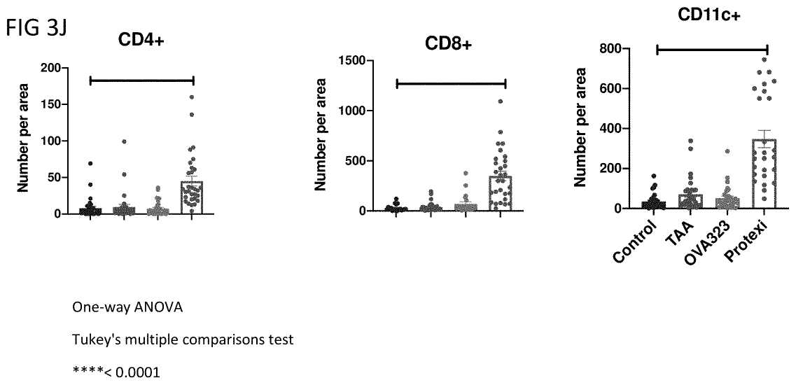

Through the two different tumor models, we have observed promising results that PROTEXI is able to enhance CD8-directed T cell response and tumor rejection. To find out the importance of non-tumor specific CD4 T cell function in PROTEXI, we decided to directly co-administered OTII (OVA323 specific) CD4 T cells with PROTEXI in the B16F10 melanoma model. The treatment groups were composed of 1) Control, 2) OTII-CD4T+DC_{TAA}, and 3) OTII-CD4T+DC_{OVA323}, 4) OTII-CD4T+PROTEXI. Following B16F10 injection, a single injection of OTII-CD4T cells (1×10⁶/inj. i.v.) and PROTEXI vaccination were conducted at d0. The co-administration of OTII-CD4T and PROTEXI dramatically enhanced B16F10 tumor rejection as compared to the group of control, CD4T+DC_{T}AA, or CD4T+DC_{OVA323}. Interestingly, CD4T+ DC_{OVA323} also showed tumor suppressive potential to the similar level of DC_{TAA} group, possibly due to the epitope spreading effect. These results strongly suggest that non-tumor specific, but highly immunogenic, CD4T epitope has a prodigious capacity to induce tumor rejection. See FIGS. 3A-3D.

### Example 6—Epitope Spreading

The emergence of M30-11/Trp2-specific T cell clones were verified in the vaccinated groups especially in the PROTEXI group with the highest expansion. Epitope-loaded Tetramer assay showed that CD8+Trp2-reactive TCR+ clones were induced in the vaccinated groups where with the PROTEXI group showed a greater expansion. Epitope spreading was evidenced by verifying T cell response to unvaccinated epitope (Luc2), expressed in the B16F10-Luc2. See FIGS. 3E-3H.

### Example 7—PROTEXI Recruits TIL in Mouse Model for Melanoma

H&E staining revealed that CD4T+PROTEXI co-treated group showed a characteristic of necrosis within the tumor (Pink stained area). CD4T+PROTEXI co-treated group predominantly recruited immense number of TIL (CD4+, CD8+, CD11c+) in the tumor compared to the other vaccinated groups. Thus, the results demonstrate that CD4T+PROTEXI remarkably empowers immune-mediated tumor rejection by leveraging non-tumor specific CD4 helper T cells, effectively driving the expansion of TAA-specific CTL and the recruitment of TIL. See FIGS. 3I-3J.

### Example 8—CD4 T Cell Depletion Abrogated PROTEXI-Mediated Tumor Rejection in Mouse Melanoma Model

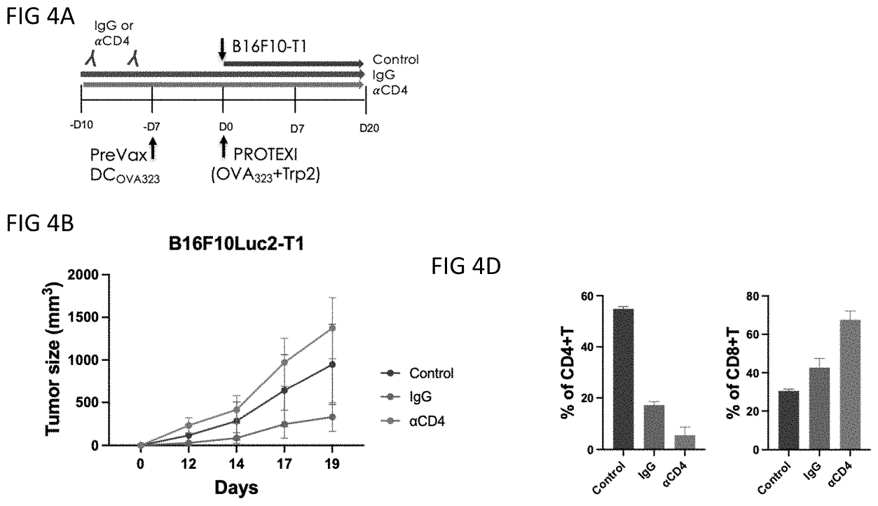

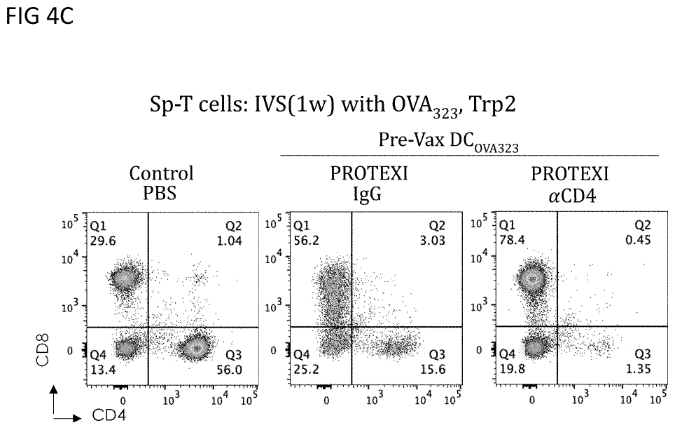

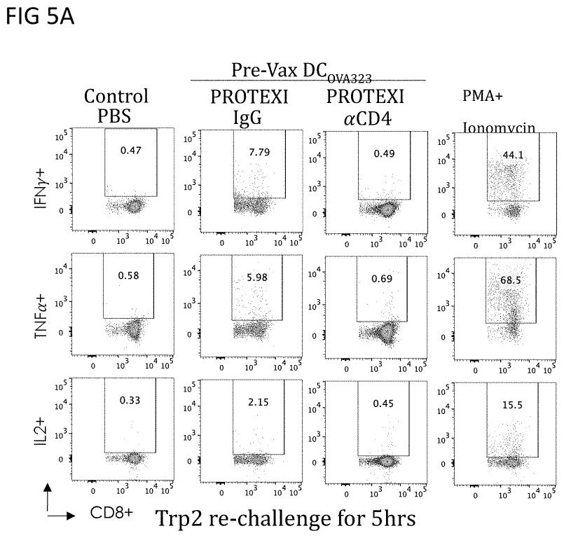

To demonstrate the importance of CD4T helper function, the CD4T cells were depleted with administration of αCD4T or IgG antibody prior to PreVax_{OVA323} and the subsequent PROTEXI(OVA₃₂₃+Trp2) vaccination. B16F10Luc2-T1 is more aggressive cell line established from the tumor grown in vivo. The control group displayed more aggressive growth whereas PreVax+PROTEXI (IgG) treatment effectively suppressed tumor growth. On the other hand, PreVax+PROTEXI (αCD4 Ab) drastically lost tumor suppressive potential due to the prior-depletion of CD4T helper cells. To look into the variance of epitope-specific T cell responses, the splenocytes of treated mice were subjected to in vitro stimulation (IVS) with OVA₃₂₃ and Trp2 peptides for a week. CD4 T cell depletion was well induced by administration of αCD4 antibody which was partially re-constituted to the level of <2% CD4T subpopulation. Interestingly, the αCD4 antibody treatment greatly shifted CD8 T cell proportion to >78% in contrast to that of IgG group (56%) and control (30%). See FIG. 4A-4D.

### Example 9—PROTEXI-Induced Trp2-Specific CD8T Cell Activation is Principally Depends on CD4 Helper T Cells

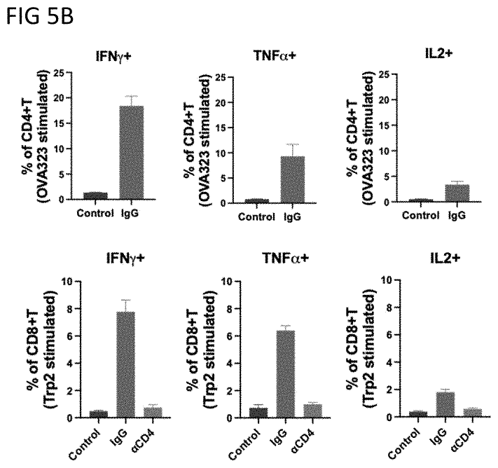

Subsequently, the epitope-specific T cell expansion in IVS splenocytes were evaluated by flow cytometric analysis after re-challenging with OVA₃₂₃ or Trp2 peptide. The multi-functional CD8T cells secreting IFNγ, TNFα, IL-2 were observed in PROTEXI-IgG group exclusively. It is important to note that CD4 T cell depletion near completely removed PROTEXI-mediated Trp2-CD8T cell activation to the level of control. The OVA323-CD4T cell activation was also verified only in the PROTEXI-IgG group. Therefore, tumor-epitope restricted CD8T cell activation and expansion in the PROTEXI group was prominently depending on the presence of CD4T helper cells. See FIG. 5A-5B.

### Example 10—CD4 T Depletion Resulted in Loss of T Cell Memory Cell Formation Induced by PROTEXI

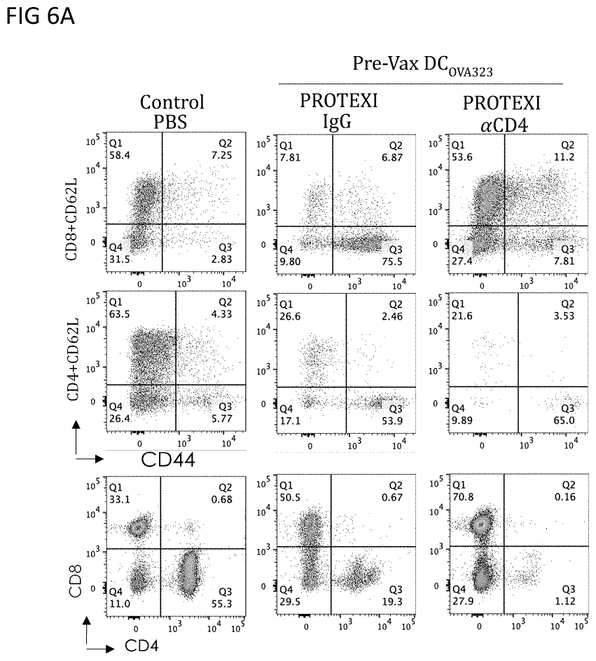

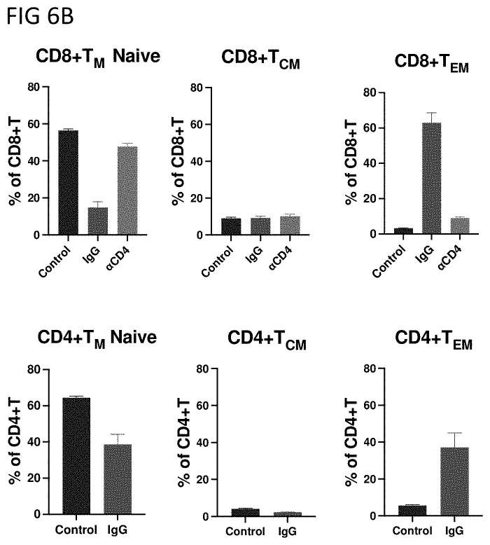

It is well documented that CD4 T cells play a pivotal role for memory T cell formation that is important for long-lasting tumor rejection and preventing relapse down the road. So, we determined that non-tumor specific CD4T cells are affecting memory T cell formation in PROTEXI vaccinated group with or without CD4 T cell depletion. The flow cytometric analysis demonstrated that IgG-PROTEXI group has significantly elevated level of CD8T effector memory cells (75% of CD8⁺CD44⁺CD62L⁻). The CD4 T cell depletion in PROTEXI, however, produced residual central memory (11% of CD8⁺CD44⁺ CD62L⁺) and effector memory CD8T cells (7.8% of CD8⁺CD44⁺CD62L⁻) while the majority turned out to be naïve CD8T memory—cells (54% of CD8⁺CD44⁻CD62L⁺) similar to the level of control. CD4T effector memory cells was also augmented in the PROTEXI+IgG group compared to control. As such, OVA₃₂₃-CD4T cells exert helper function during CD4/CD8T effector memory T cell formation which would contribute to long-term tumor regression without relapse. See FIG. 6A-6B.

### Example 11—PROTEXI Robustly Inhibited Mouse Model of Osteosarcoma Growth by Exploiting Spike Epitope-Specific Human CD4T Cells

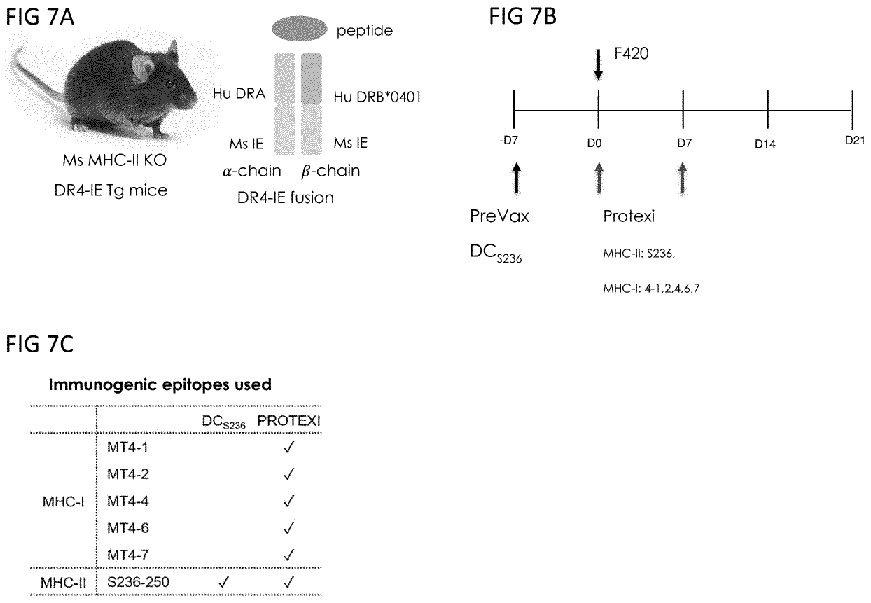

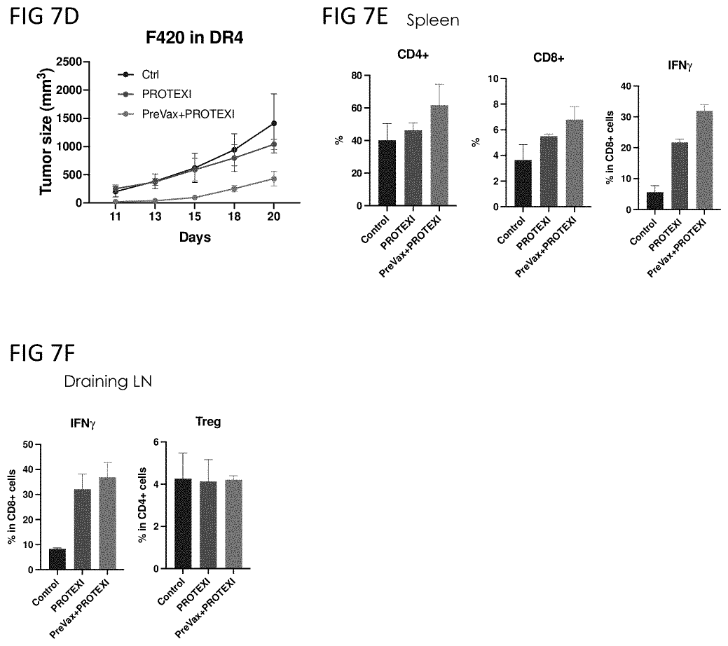

Further, to see the effect of PROTEXI loaded with Spike epitope, we utilized humanized mouse model expressing HLA-DRB4. The treatment groups were composed of 1) Control, 2) PROTEXI, and 3) PreVax+PROTEXI. The mice in PreVax group were vaccinated (1×10⁶/inj. SQ) with DC pulsed a HLA-DRB*0401-restricted Spike epitope (S236) at −d7, followed by two-time injection of PROTEXI (1×10⁶/inj. SQ) pulsed with S236, and CD8-specific epitopes (MT4-1, 2, 4, 6, 7). The mice in PreVax+PROTEXI group showed immune-mediated F420 rejection in humanized (DR4) mice. Although CD8+ T cell subset were unusually low in DR4 mice, PreVax+PROTEXI increased 5 or 6-fold of IFN-γ+CD8+ T cells in spleen and draining lymph nodes. Thus, PROTEXI loaded with poorly immunogenic NeoAgs showed F420 osteosarcoma rejection in humanized mice by leveraging DRB*0401-restricted Spike (S236) epitope, suggesting that PROTEXI could be a novel cancer vaccine in the clinical settings by which largely improve anti-tumor immunity of poorly immunogenic NeoAgs especially for low TMB cancers including sarcomas. See FIGS. 7A-7F.

### Example 12—Vaccination Against Cancer

Subcutaneous autologous DC vaccine loaded with SARS-CoV-2 epitopes and Cancer Testis Antigen peptides is given to patients with high-risk sarcoma.

Target condition may be osteosarcoma, Synovial Cell Sarcoma, Mixoid/round Liposarcoma, or Ewing's Sarcoma.

Autologous DC Vaccine (PROTEXI™) simultaneously loaded with SARS-CoV-2 epitopes and CTA peptides derived from MAGE-A1, A3, A4, NY-ESO-1, PRAME are used.

Subcutaneous autologous personalized neoantigen DC vaccine loaded (PROTEXI) is given in combination with Vactosertib. Five cycles of PROTEXI is given with vaccine administered by subcutaneous injection on day 2 of weeks 1, 5, 9, 13, 17 along with Vactosertib treatment (200-340 mg thrice daily oral administration, administered as weekly cycles of 5 days on drug and two days off the drug for four-week cycles). Responders will be eligible to receive two additional doses of PROTEXI as a maintenance vaccine, which is administered two months apart.

Method of treatment includes collection of tumor tissue (a minimum of 30 mg) and normal peripheral blood mononuclear cells (PBMCS, isolated from 20 ml of peripheral blood) for neoantigen identification, using a proprietary neoantigen discovery platform; and evaluating their baseline level of response to tumor neoantigens defined by the ex vivo response of PBMCs to the tumor-specific neoantigens, Spike epitopes, and/or cancer/testis antigen, followed by administering the autologous, neoantigen dendritic cell vaccine (PROTEXI), optionally in combination with Vactosertib or immune check point inhibitor.

### REFERENCES

1. Ferguson, J. L. and S. P. Turner, Bone Cancer: Diagnosis and Treatment Principles. Am Fam Physician, 2018. 98(4): p. 205-213.
2. Katz, D., E. Palmerini, and S. M. Pollack, More Than 50 Subtypes of Soft Tissue Sarcoma: Paving the Path for Histology-Driven Treatments. Am Soc Clin Oncol Educ Book, 2018. 38: p. 925-938.
3. Mitchell, G., S. M. Pollack, and M. J. Wagner, Targeting cancer testis antigens in synovial sarcoma. J Immunother Cancer, 2021. 9(6).
4. Tay, R. E., E. K. Richardson, and H. C. Toh, Revisiting the role of CD4(+) T cells in cancer immunotherapy-new insights into old paradigms. Cancer Gene Ther, 2021. 28(1-2): p. 5-17.
5. Ossendorp, F., et al., Specific T helper cell requirement for optimal induction of cytotoxic T lymphocytes against major histocompatibility complex class II negative tumors. J Exp Med, 1998. 187(5): p. 693-702.
6. Alspach, E., et al., MHC-II neoantigens shape tumour immunity and response to immunotherapy. Nature, 2019. 574(7780): p. 696-701.
7. Hu, Z., et al., Personal neoantigen vaccines induce persistent memory T cell responses and epitope spreading in patients with melanoma. Nat Med, 2021. 27(3): p. 515-525.
8. Knutson, K. L. and M. L. Disis, Tumor antigen-specific T helper cells in cancer immunity and immunotherapy. Cancer Immunol Immunother, 2005. 54(8): p. 721-8.
9. Menares, E., et al., Tissue-resident memory CD8(+) T cells amplify anti-tumor immunity by triggering antigen spreading through dendritic cells. Nat Commun, 2019. 10(1): p. 4401.
10. Beck, J. D., et al., mRNA therapeutics in cancer immunotherapy. Mol Cancer, 2021. 20(1): p. 69.
11. D'Angelo, S. P., et al., Nivolumab with or without ipilimumab treatment for metastatic sarcoma (Alliance A091401): two open-label, non-comparative, randomised, phase 2 trials. Lancet Oncol, 2018. 19(3): p. 416-426.
12. Kumar, C., et al., Substantial remission of prostate adenocarcinoma with dendritic cell therapy APCEDEN® in combination with chemotherapy. Future Science Oa, 2019. 5(10).
13. Sharan, B., et al., Substantial tumor regression in prostate cancer patient with extensive skeletal metastases upon Immunotherapy (APCEDEN): A case report. Medicine (Baltimore), 2020. 99(8): p. e18889.
14. Castle, J. C., et al., Exploiting the mutanome for tumor vaccination. Cancer Res, 2012. 72(5): p. 1081-91.
15. Blass, E. and P. A. Ott, Advances in the development of personalized neoantigen-based therapeutic cancer vaccines. Nat Rev Clin Oncol, 2021. 18(4): p. 215-229.
16. Zhang, R., et al., Personalized neoantigen-pulsed dendritic cell vaccines show superior immunogenicity to neoantigen-adjuvant vaccines in mouse tumor models. Cancer Immunol Immunother, 2020. 69(1): p. 135-145.
17. Hyo Song Kim, J.-H. A., Jeong Eun Kim, Jung Yong Hong, Jeeyun Lee, Sung Hyun Kim, Jin Lee, Sunjin Hwang, Min Kyung Jeon, Seong-Jin Kim, A phase I study of TGF-β inhibitor, vactosertib in combination with imatinib in patients with advanced desmoid tumor (aggressive fibromatosis). Journal of Clinical Oncology 2020. 38: p. 11557-11557.
18. Leung, S., et al., OT-II TCR transgenic mice fail to produce anti-ovalbumin antibodies upon vaccination. Cell Immunol, 2013. 282(2): p. 79-84.
19. Touloukian, C. E., et al., Identification of a MHC class II-restricted human gp100 epitope using DR4-IE transgenic mice. J Immunol, 2000. 164(7): p. 3535-42.
20. Zhao, S., et al., NKD2, a negative regulator of Wnt signaling, suppresses tumor growth and metastasis in osteosarcoma. Oncogene, 2015. 34(39): p. 5069-79.

All of the references cited herein are incorporated by reference in their entirety.

Those skilled in the art will recognize, or be able to ascertain using no more than routine experimentation, many equivalents to the specific embodiments of the invention specifically described herein.

1. A method of treating cancer, comprising administering to a person suffering from cancer or in remission from cancer, an antigen presenting cell loaded with an immunogenic CD4 T cell activating antigen and a CD8 T cell activating neoantigen specific for the cancer.

2. The method of claim 1, wherein the antigen presenting cell is dendritic cell.

3. The method of claim 1, wherein, the antigen presenting cell is autologous.

4. The method of claim 1, wherein the CD4 T cell activating antigen is a peptide.

5. The method of claim 4, wherein the peptide is a fragment of a pathogen, or epitope fragments used for preventive vaccination throughout lifetime.

6. The method of claim 5, wherein the pathogen is a bacteria, virus, or parasite.

7. The method of claim 6, wherein the virus is coronavirus, Influenza, Mycobacterium tuberculosis, Cytomegalovirus(CMV).

8. The method of claim 7, wherein the virus is coronavirus.

9. The method of claim 4, wherein the peptide is a fragment of spike protein, ORF3a, ORF7a, ORF6, ORFS, nsp2, nsp5 of coronavirus, HA of influenza, GlfT2, fas, fbpA, iniB, PPE15 of M. tuberculosis, pp50, pp65, IE-1, gB, gH of CMV.

10. The method of claim 1, wherein the CD8 T cell activating neoantigen is a publicly known neoantigen.

11. The method of claim 10, wherein the neoantigen is any peptide from Tables 4 to 7.

12. The method of claim 11, wherein the neoantigen is a cancer/testis antigen.

13. The method of claim 1, wherein the CD8 T cell activating neoantigen is personalized.

14. The method of claim 1, comprising checking for presence of preexisting immune response, and administering the antigen presenting cell loaded with an immunogenic CD4 T cell activating antigen and a CD8 T cell activating neoantigen specific for the cancer to the person in need thereof.

15. The method of claim 1, wherein the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof.

16. The method of claim 15, wherein the cancer is sarcoma.

17. The method of claim 16, wherein the sarcoma is osteosarcoma.

18. A method of enhancing anti-tumor immunity of a person in remission of cancer, comprising administering to the person an antigen presenting cell loaded with an immunogenic CD4 T cell activating antigen and a CD8 T cell activating neoantigen specific for the cancer.

19. The method of claim 18, wherein the antigen presenting cell is dendritic cell.

20. The method of claim 18, wherein, the antigen presenting cell is autologous.

21. The method of claim 18, wherein the CD4 T cell activating antigen is a peptide.

22. The method of claim 21, wherein the peptide is a fragment of a pathogen, or epitope fragments used for preventive vaccination throughout lifetime.

23. The method of claim 22, wherein the pathogen is a bacteria, virus, or parasite.

24. The method of claim 23, wherein the virus is coronavirus, Influenza, Mycobacterium tuberculosis, Cytomegalovirus(CMV).

25. The method of claim 24, wherein the virus is coronavirus.

26. The method of claim 21, wherein the peptide is a fragment of spike protein, ORF3a, ORF7a, ORF6, ORFS, nsp2, nsp5 of coronavirus, HA of influenza, GlfT2, fas, fbpA, iniB, PPE15 of M. tuberculosis, pp50, pp65, IE-1, gB, gH of CMV

27. The method of claim 18, wherein the CD8 T cell activating neoantigen is a publicly known neoantigen.

28. The method of claim 27, wherein the neoantigen is any peptide from Tables 4 to 7.

29. The method of claim 28, wherein the neoantigen is a cancer/testis antigen.

30. The method of claim 18, wherein the CD8 T cell activating neoantigen is personalized.

31. The method of claim 18, comprising checking for presence of preexisting immune response, and administering the antigen presenting cell loaded with an immunogenic CD4 T cell activating antigen and a CD8 T cell activating neoantigen specific for the cancer to the person in need thereof.

32. The method of claim 18, wherein the cancer is prostate cancer, breast cancer, bladder cancer, lung cancer, colorectal cancer, pancreatic cancer, liver cancer, renal cancer, renal cell carcinoma, melanoma, sarcoma, head and neck cancer, glioblastoma, or a combination thereof.

33. The method of claim 32, wherein the cancer is sarcoma.

34. The method of claim 33, wherein the sarcoma is osteosarcoma.

35. A cancer vaccine, comprising an antigen-presenting cell co-presenting a non-tumor-specific, but highly immunogenic CD4 T cell epitope and tumor-specific CD8 T cell neoepitopes simultaneously, which empowers antitumor immune response by engaging CD4 T cells for activation of CD8 T cells.

36. The vaccine of claim 35, which is autologous with respect to the subject treated.

37. The vaccine of claim 35, wherein the antigen presenting cell is dendritic cell.

38. The vaccine of claim 35, wherein CD4 T cell epitope derives from bacteria or virus.

39. The vaccine of claim 38, wherein the CD4 T cell epitope is spike protein from coronavirus.
